SEQUENCE LISTING (65 sequences)

SEQ ID NO: 1 - DNA, 1371 bp, synthetic construct
  misc_feature 1..1371: note = Synthetic
  source 1..1371: mol_type = other DNA
caggtgcagctggtggagtctgggggaggcgtagtccagcctgggaggtccctgagactctcctgtgcagcgtctggattcaccttcagtgcgtattggatgcactgggtccgccaggctccaggcaaggggctggagtgggtggcagttatatcgtatgatggaagtaataaatactatgcagactccgtgaagggccgattcaccatctccagagaccattccaagaacacgctgtatctgcaaatgaacagcctgagagccgaggacacggcggtgtactactgcgcaaaaggggacgcctacgactcctggaggatactactcggcggagattactacggcatggatgtttggggccagggaacaactgtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 2 - protein, 456 aa, synthetic construct
  REGION 1..456: note = Synthetic
QVQLVESGGGVVQPGRSLRLSCAASGFTFSAYWMHWVRQAPGKGLEWVAVISYDGSNKYYADSVKGRFTISRDHSKNTLYLQMNSLRAEDTAVYYCAKGDAYDSWRILLGGDYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - DNA, 651 bp, synthetic construct
  misc_feature 1..651: note = Synthetic
  source 1..651: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggcccctcatctatggcgcatccagcagggccactggcatcccagacagattcagtggcagtgggtctgggacagacttcactctcaccatcagcagactggagcctgaagattttgcagtgtattactgtcagcaggggggggacgcccctccttacactttcggcggagggaccaaggttgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 4 - protein, 216 aa, synthetic construct
  REGION 1..216: note = Synthetic
EIVLTQSPGTLSLSPGERATLSCRASQSVSSSYLAWYQQKPGQAPRPLIYGASSRATGIPDRFSGSGSGTDFTLTISRLEPEDFAVYYCQQGGDAPPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 1362 bp, synthetic construct
  misc_feature 1..1362: note = Synthetic
  source 1..1362: mol_type = other DNA
cagctgcagctgcaggagtcgggcccaggactggtgaagccttcggagaccctgtccctcacctgcactgtctctggtggctccatcacgagtagtagttactactgggcgtggatccgccagcccccagggaaggggctggagtggattgggaagatctattatagtgggagcacccattacaacccgtccctcaagagtcgagtcaccatatccgtagacacgtccaagaaccagttctccctgaagttgagttctgtgaccgccgcagacacggcggtgtactactgcgccagagttggaggacacggctacggagactccgcagggttagccttcgatatctggggtcagggtacaatggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 6 - protein, 453 aa, synthetic construct
  REGION 1..453: note = Synthetic
QLQLQESGPGLVKPSETLSLTCTVSGGSITSSSYYWAWIRQPPGKGLEWIGKIYYSGSTHYNPSLKSRVTISVDTSKNQFSLKLSSVTAADTAVYYCARVGGHGYGDSAGLAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - DNA, 648 bp, synthetic construct
  misc_feature 1..648: note = Synthetic
  source 1..648: mol_type = other DNA
gaaattgtgttgacgcagtctccaggcaccctgtctttgtctccaggggaaagagccaccctctcctgcagggccagtcagagtgttagcagcgccttcttagcctggtaccagcagaaacctggccaggctcccaggctcctcatctatggtgcatcctacaggcacactggcatcccagacaggttcagtggcagtgggtctgggacagacttcactctcaccatcagcagactggagcctgaagattttgcagtgtattactgtcagcactacggagttggtcctaggactttcggcggagggaccaaggttgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 8 - protein, 215 aa, synthetic construct
  REGION 1..215: note = Synthetic
EIVLTQSPGTLSLSPGERATLSCRASQSVSSAFLAWYQQKPGQAPRLLIYGASYRHTGIPDRFSGSGSGTDFTLTISRLEPEDFAVYYCQHYGVGPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - DNA, 1356 bp, synthetic construct
  misc_feature 1..1356: note = Synthetic
  source 1..1356: mol_type = other DNA
caggtgcagctggtgcagtctggggctgaggtgaaggagcctggggcctcagtgaaggtttcctgcaaggcatctggatacaccttcgcggagtactatatgcactgggtgcgacaggcccctggacaagggcttgagtggatgggagcgattaaccctagtggtagtcatacaagctacgcacagaagttccagggcagagtcaccatgaccagggacacgtccacgagcacagtctacatggagctgagcagcctgagatctgaggacacggcggtgtactactgcgctagaagtattagtggcaaacgaggtggcgattactacggcatggatgtttggggccagggaacaactgtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 10 - protein, 451 aa, synthetic construct
  REGION 1..451: note = Synthetic
QVQLVQSGAEVKEPGASVKVSCKASGYTFAEYYMHWVRQAPGQGLEWMGAINPSGSHTSYAQKFQGRVTMTRDTSTSTVYMELSSLRSEDTAVYYCARSISGKRGGDYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - DNA, 645 bp, synthetic construct
  misc_feature 1..645: note = Synthetic
  source 1..645: mol_type = other DNA
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaagcctccagcttggaaagtggggtcccatcaaggttcagcggcagtggatctgggacagaattcactctcaccatcagcagcctgcagcctgatgattttgcaacttattactgccagcagtacagcatcttccctttcacttttggcggagggaccaaggttgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 12 - protein, 214 aa, synthetic construct
  REGION 1..214: note = Synthetic
DIQMTQSPSTLSASVGDRVTITCRASQSISSWLAWYQQKPGKAPKLLIYKASSLESGVPSRFSGSGSGTEFTLTISSLQPDDFATYYCQQYSIFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 387 bp, synthetic construct
  misc_feature 1..387: note = Synthetic
  source 1..387: mol_type = other DNA
caggtgcagctggtggagtctgggggaggcgtagtccagcctgggaggtccctgagactctcctgtgcagcgtctggattcaccttcagtgcgtattggatgcactgggtccgccaggctccaggcaaggggctggagtgggtggcagttatatcgtatgatggaagtaataaatactatgcagactccgtgaagggccgattcaccatctccagagaccattccaagaacacgctgtatctgcaaatgaacagcctgagagccgaggacacggcggtgtactactgcgcaaaaggggacgcctacgactcctggaggatactactcggcggagattactacggcatggatgtttggggccagggaacaactgtcaccgtctcctca

SEQ ID NO: 14 - protein, 129 aa, synthetic construct
  REGION 1..129: note = Synthetic
QVQLVESGGGVVQPGRSLRLSCAASGFTFSAYWMHWVRQAPGKGLEWVAVISYDGSNKYYADSVKGRFTISRDHSKNTLYLQMNSLRAEDTAVYYCAKGDAYDSWRILLGGDYYGMDVWGQGTTVTVSS

SEQ ID NO: 15 - DNA, 24 bp, synthetic construct
  misc_feature 1..24: note = Synthetic
  source 1..24: mol_type = other DNA
ggattcaccttcagtgcgtattgg

SEQ ID NO: 16 - protein, 8 aa, synthetic construct
  REGION 1..8: note = Synthetic
GFTFSAYW

SEQ ID NO: 17 - DNA, 24 bp, synthetic construct
  misc_feature 1..24: note = Synthetic
  source 1..24: mol_type = other DNA
atatcgtatgatggaagtaataaa

SEQ ID NO: 18 - protein, 8 aa, synthetic construct
  REGION 1..8: note = Synthetic
ISYDGSNK

SEQ ID NO: 19 - DNA, 66 bp, synthetic construct
  misc_feature 1..66: note = Synthetic
  source 1..66: mol_type = other DNA
gcaaaaggggacgcctacgactcctggaggatactactcggcggagattactacggcatggatgtt

SEQ ID NO: 20 - protein, 22 aa, synthetic construct
  REGION 1..22: note = Synthetic
AKGDAYDSWRILLGGDYYGMDV

SEQ ID NO: 21 - DNA, 327 bp, synthetic construct
  misc_feature 1..327: note = Synthetic
  source 1..327: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggcccctcatctatggcgcatccagcagggccactggcatcccagacagattcagtggcagtgggtctgggacagacttcactctcaccatcagcagactggagcctgaagattttgcagtgtattactgtcagcaggggggggacgcccctccttacactttcggcggagggaccaaggttgagatcaaa

SEQ ID NO: 22 - protein, 109 aa, synthetic construct
  REGION 1..109: note = Synthetic
EIVLTQSPGTLSLSPGERATLSCRASQSVSSSYLAWYQQKPGQAPRPLIYGASSRATGIPDRFSGSGSGTDFTLTISRLEPEDFAVYYCQQGGDAPPYTFGGGTKVEIK

SEQ ID NO: 23 - DNA, 21 bp, synthetic construct
  misc_feature 1..21: note = Synthetic
  source 1..21: mol_type = other DNA
cagagtgttagcagcagctac

SEQ ID NO: 24 - protein, 7 aa, synthetic construct
  REGION 1..7: note = Synthetic
QSVSSSY

SEQ ID NO: 25-26 - skipped (2 entries)

SEQ ID NO: 27 - DNA, 30 bp, synthetic construct
  misc_feature 1..30: note = Synthetic
  source 1..30: mol_type = other DNA
cagcaggggggggacgcccctccttacact

SEQ ID NO: 28 - protein, 10 aa, synthetic construct
  REGION 1..10: note = Synthetic
QQGGDAPPYT

SEQ ID NO: 29 - DNA, 378 bp, synthetic construct
  misc_feature 1..378: note = Synthetic
  source 1..378: mol_type = other DNA
cagctgcagctgcaggagtcgggcccaggactggtgaagccttcggagaccctgtccctcacctgcactgtctctggtggctccatcacgagtagtagttactactgggcgtggatccgccagcccccagggaaggggctggagtggattgggaagatctattatagtgggagcacccattacaacccgtccctcaagagtcgagtcaccatatccgtagacacgtccaagaaccagttctccctgaagttgagttctgtgaccgccgcagacacggcggtgtactactgcgccagagttggaggacacggctacggagactccgcagggttagccttcgatatctggggtcagggtacaatggtcaccgtctcctca

SEQ ID NO: 30 - protein, 126 aa, synthetic construct
  REGION 1..126: note = Synthetic
QLQLQESGPGLVKPSETLSLTCTVSGGSITSSSYYWAWIRQPPGKGLEWIGKIYYSGSTHYNPSLKSRVTISVDTSKNQFSLKLSSVTAADTAVYYCARVGGHGYGDSAGLAFDIWGQGTMVTVSS

SEQ ID NO: 31 - DNA, 30 bp, synthetic construct
  misc_feature 1..30: note = Synthetic
  source 1..30: mol_type = other DNA
ggtggctccatcacgagtagtagttactac

SEQ ID NO: 32 - protein, 10 aa, synthetic construct
  REGION 1..10: note = Synthetic
GGSITSSSYY

SEQ ID NO: 33 - DNA, 21 bp, synthetic construct
  misc_feature 1..21: note = Synthetic
  source 1..21: mol_type = other DNA
atctattatagtgggagcacc

SEQ ID NO: 34 - protein, 7 aa, synthetic construct
  REGION 1..7: note = Synthetic
IYYSGST

SEQ ID NO: 35 - DNA, 54 bp, synthetic construct
  misc_feature 1..54: note = Synthetic
  source 1..54: mol_type = other DNA
gccagagttggaggacacggctacggagactccgcagggttagccttcgatatc

SEQ ID NO: 36 - protein, 18 aa, synthetic construct
  REGION 1..18: note = Synthetic
ARVGGHGYGDSAGLAFDI

SEQ ID NO: 37 - DNA, 324 bp, synthetic construct
  misc_feature 1..324: note = Synthetic
  source 1..324: mol_type = other DNA
gaaattgtgttgacgcagtctccaggcaccctgtctttgtctccaggggaaagagccaccctctcctgcagggccagtcagagtgttagcagcgccttcttagcctggtaccagcagaaacctggccaggctcccaggctcctcatctatggtgcatcctacaggcacactggcatcccagacaggttcagtggcagtgggtctgggacagacttcactctcaccatcagcagactggagcctgaagattttgcagtgtattactgtcagcactacggagttggtcctaggactttcggcggagggaccaaggttgagatcaaa

SEQ ID NO: 38 - protein, 108 aa, synthetic construct
  REGION 1..108: note = Synthetic
EIVLTQSPGTLSLSPGERATLSCRASQSVSSAFLAWYQQKPGQAPRLLIYGASYRHTGIPDRFSGSGSGTDFTLTISRLEPEDFAVYYCQHYGVGPRTFGGGTKVEIK

SEQ ID NO: 39 - DNA, 21 bp, synthetic construct
  misc_feature 1..21: note = Synthetic
  source 1..21: mol_type = other DNA
cagagtgttagcagcgccttc

SEQ ID NO: 40 - protein, 7 aa, synthetic construct
  REGION 1..7: note = Synthetic
QSVSSAF

SEQ ID NO: 41-42 - skipped (2 entries)

SEQ ID NO: 43 - DNA, 27 bp, synthetic construct
  misc_feature 1..27: note = Synthetic
  source 1..27: mol_type = other DNA
cagcactacggagttggtcctaggact

SEQ ID NO: 44 - protein, 9 aa, synthetic construct
  REGION 1..9: note = Synthetic
QHYGVGPRT

SEQ ID NO: 45 - DNA, 372 bp, synthetic construct
  misc_feature 1..372: note = Synthetic
  source 1..372: mol_type = other DNA
caggtgcagctggtgcagtctggggctgaggtgaaggagcctggggcctcagtgaaggtttcctgcaaggcatctggatacaccttcgcggagtactatatgcactgggtgcgacaggcccctggacaagggcttgagtggatgggagcgattaaccctagtggtagtcatacaagctacgcacagaagttccagggcagagtcaccatgaccagggacacgtccacgagcacagtctacatggagctgagcagcctgagatctgaggacacggcggtgtactactgcgctagaagtattagtggcaaacgaggtggcgattactacggcatggatgtttggggccagggaacaactgtcaccgtctcctca

SEQ ID NO: 46 - protein, 124 aa, synthetic construct
  REGION 1..124: note = Synthetic
QVQLVQSGAEVKEPGASVKVSCKASGYTFAEYYMHWVRQAPGQGLEWMGAINPSGSHTSYAQKFQGRVTMTRDTSTSTVYMELSSLRSEDTAVYYCARSISGKRGGDYYGMDVWGQGTTVTVSS

SEQ ID NO: 47 - DNA, 24 bp, synthetic construct
  misc_feature 1..24: note = Synthetic
  source 1..24: mol_type = other DNA
ggatacaccttcgcggagtactat

SEQ ID NO: 48 - protein, 8 aa, synthetic construct
  REGION 1..8: note = Synthetic
GYTFAEYY

SEQ ID NO: 49 - DNA, 24 bp, synthetic construct
  misc_feature 1..24: note = Synthetic
  source 1..24: mol_type = other DNA
attaaccctagtggtagtcataca

SEQ ID NO: 50 - protein, 8 aa, synthetic construct
  REGION 1..8: note = Synthetic
INPSGSHT

SEQ ID NO: 51 - DNA, 51 bp, synthetic construct
  misc_feature 1..51: note = Synthetic
  source 1..51: mol_type = other DNA
gctagaagtattagtggcaaacgaggtggcgattactacggcatggatgtt

SEQ ID NO: 52 - protein, 17 aa, synthetic construct
  REGION 1..17: note = Synthetic
ARSISGKRGGDYYGMDV

SEQ ID NO: 53 - DNA, 321 bp, synthetic construct
  misc_feature 1..321: note = Synthetic
  source 1..321: mol_type = other DNA
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaagcctccagcttggaaagtggggtcccatcaaggttcagcggcagtggatctgggacagaattcactctcaccatcagcagcctgcagcctgatgattttgcaacttattactgccagcagtacagcatcttccctttcacttttggcggagggaccaaggttgagatcaaa

SEQ ID NO: 54 - protein, 107 aa, synthetic construct
  REGION 1..107: note = Synthetic
DIQMTQSPSTLSASVGDRVTITCRASQSISSWLAWYQQKPGKAPKLLIYKASSLESGVPSRFSGSGSGTEFTLTISSLQPDDFATYYCQQYSIFPFTFGGGTKVEIK

SEQ ID NO: 55 - DNA, 18 bp, synthetic construct
  misc_feature 1..18: note = Synthetic
  source 1..18: mol_type = other DNA
cagagtattagtagctgg

SEQ ID NO: 56 - protein, 6 aa, synthetic construct
  REGION 1..6: note = Synthetic
QSISSW

SEQ ID NO: 57-58 - skipped (2 entries)

SEQ ID NO: 59 - DNA, 27 bp, synthetic construct
  misc_feature 1..27: note = Synthetic
  source 1..27: mol_type = other DNA
cagcagtacagcatcttccctttcact

SEQ ID NO: 60 - protein, 9 aa, synthetic construct
  REGION 1..9: note = Synthetic
QQYSIFPFT

SEQ ID NO: 61 - protein, 509 aa, synthetic construct
  REGION 1..509: note = Human ACVR1 Accession Q04771
MVDGVMILPVLIMIALPSPSMEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLEVGLIILSVVFAVCLLACLLGVALRKFKRRNQERLNPRDVEYGTIEGLITTNVGDSTLADLLDHSCTSGSGSGLPFLVQRTVARQITLLECVGKGRYGEVWRGSWQGENVAVKIFSSRDEKSWFRETELYNTVMLRHENILGFIASDMTSRHSSTQLWLITHYHEMGSLYDYLQLTTLDTVSCLRIVLSIASGLAHLHIEIFGTQGKPAIAHRDLKSKNILVKKNGQCCIADLGLAVMHSQSTNQLDVGNNPRVGTKRYMAPEVLDETIQVDCFDSYKRVDIWAFGLVLWEVARRMVSNGIVEDYKPPFYDVVPNDPSFEDMRKVVCVDQQRPNIPNRWFSDPTLTSLAKLMKECWYQNPSARLTALRIKKTLTKIDNSLDKLKTDC

SEQ ID NO: 62 - protein, 509 aa, synthetic construct
  REGION 1..509: note = Mouse ACVR1 Accession P37172
MVDGVMILPVLMMMAFPSPSVEDEKPKVNQKLYMCVCEGLSCGNEDHCEGQQCFSSLSINDGFHVYQKGCFQVYEQGKMTCKTPPSPGQAVECCQGDWCNRNITAQLPTKGKSFPGTQNFHLEVGLIILSVVFAVCLLACILGVALRKFKRRNQERLNPRDVEYGTIEGLITTNVGDSTLAELLDHSCTSGSGSGLPFLVQRTVARQITLLECVGKGRYGEVWRGSWQGENVAVKIFSSRDEKSWFRETELYNTVMLRHENILGFIASDMTSRHSSTQLWLITHYHEMGSLYDYLQLTTLDTVSCLRIVLSIASGLAHLHIEIFGTQGKSAIAHRDLKSKNILVKKNGQCCIADLGLAVMHSQSTNQLDVGNNPRVGTKRYMAPEVLDETIQVDCFDSYKRVDIWAFGLVLWEVARRMVSNGIVEDYKPPFYDVVPNDPSFEDMRKVVCVDQQRPNIPNRWFSDPTLTSLAKLMKECWYQNPSARLTALRIKKTLTKIDNSLDKLKTDC

SEQ ID NO: 63 - protein, 131 aa, synthetic construct
  REGION 1..131: note = Amino acids 1-103: Human ACVR1 extracellular domain (21-123 of accession number Q04771) Amino acids 104-131: Myc-myc-hexahistidine tag
MEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLEEQKLISEEDLGGEQKLISEEDLHHHHHH

SEQ ID NO: 64 - protein, 336 aa, synthetic construct
  REGION 1..336: note = Amino acids 1-103: Human ACVR1 extracellular domain (21-123 of accession number Q04771) Amino acids 104-336: mFc
MEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLEEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 65 - protein, 131 aa, synthetic construct
  REGION 1..131: note = Amino acids 1-103: Mouse ACVR1 extracellular domain (21-123 of accession number P37172) Amino acids 104-131: Myc-myc-hexahistidine tag
VEDEKPKVNQKLYMCVCEGLSCGNEDHCEGQQCFSSLSINDGFHVYQKGCFQVYEQGKMTCKTPPSPGQAVECCQGDWCNRNITAQLPTKGKSFPGTQNFHLEEQKLISEEDLGGEQKLISEEDLHHHHHH

(19) United States
(12) Patent Application Publication
(10) Pub. No.: US 2024/0150474 A1
(43) Pub. Date: May 9, 2024

(54) ANTI-ACVRI ANTIBODIES AND THEIR USE IN THE TREATMENT OF TRAUMA-INDUCED HETEROTOPIC OSSIFICATION

(71) Applicant: Regeneron Pharmaceuticals, Inc., Tarrytown, NY (US)

(72) Inventors: Sarah J. Hatsell, Piermont, NY (US); Vincent J. Idone, Ridgefield, CT (US); Aristides N. Economides, Tarrytown, NY (US)

(21) Appl. No.: 18/495,477

(22) Filed: Oct. 26, 2023

Related U.S. Application Data
(60) Provisional application No. 63/381,245, filed on Oct. 27, 2022.

(51) Int. Cl. C07K 16/28; A61P 19/08

(52) U.S. Cl. CPC C07K 16/2863; A61P 19/08; A61K 2039/505; C07K 2317/565; C07K 2317/92

(57) ABSTRACT

The present invention provides monoclonal antibodies and antigen-binding fragments thereof that bind to the Activin A type I receptor (ACVR1) protein, and methods of use thereof. In various embodiments of the invention, the antibodies are fully human antibodies that bind to ACVR1. In some embodiments, the antibodies of the invention and antigen-binding fragments thereof are useful for inhibiting ACVR1-mediated bone morphogenetic protein (BMP) signal transduction, thus providing a means of treating or preventing a disease, disorder or condition associated with ACVR1.

### CROSS-REFERENCES TO RELATED APPLICATIONS

This application claims the benefit of priority to U.S. Provisional Application Ser. No. 63/381,245, filed Oct. 27, 2022, which is incorporated by reference herein in its entirety.

### REFERENCE TO A SEQUENCE LISTING

The instant application contains a Sequence Listing, which is being submitted electronically in XML format and is hereby incorporated by reference in its entirety. Said XML file, created on Dec. 26, 2023, is named Sequence-Listing-40848-0115USU1.xml and is 88,347 bytes in size.

### FIELD OF THE INVENTION

The present invention is related to antibodies and antigen-binding fragments of antibodies that specifically bind to Activin A receptor type 1 (ACVR1) and/or ACVR1 mutant proteins, and therapeutic and diagnostic methods of using those antibodies.

### BACKGROUND OF THE INVENTION

Activin A receptor type 1 (ACVR1; also known as ActR1; or Activin receptor-like kinase 2; ALK2) is a single-pass transmembrane receptor, and a member of the type I Bone Morphogenic Protein (BMP) receptor of the TGF-13 receptor super family. Upon ligand binding, ACVR1 together with a type II receptor initiates a downstream signaling cascade leading to activation of receptor specific R-SMAD protein (SMAD1, SMAD5, or SMAD8) which then associates with SMAD4, leading to transcriptional regulation of genes (Massague 1998, Massaque et al. 2005).

Heterotopic ossification (HO) is a common complication associated with post-traumatic healing in clinical conditions such as fractures, spinal cord injury, traumatic brain injury, blast injuries, severe burns, and extensive surgeries such as hip arthroplasty, acetabular, and elbow surgeries. In addition to trauma-induced heterotopic ossification, this pathological phenomenon is also seen in a rare genetic disorder associated with certain mutations in ACVR1 gene.

Mutations in ACVR1 gene which encodes the BMP type I receptor ALK2, also known as ACVR1 protein, may cause fibroplasia ossificans progressiva (FOP), a rare disorder leading to progressive ectopic bone formation in soft tissues with severe impairment of body movements because of extraskeletal bone bridges. ACVR1 mutations responsible for FOP cause dysregulation of SMAD-dependent downstream signaling and confer to the mutated receptor the ability to respond to noncanonical ligand, Activin A, triggering ectopic bone formation. Gain of function mutations in the gene encoding ACVR1 lead to debilitating disorders of extra-skeletal (heterotopic) ossification in humans such as FOP. For example, the typical FOP patient may have the amino acid arginine substituted for the amino acid histidine at position 206 of ACVR1 protein. This causes a change in glycine-serine activation domain of the protein, which converts an Acvr1:Activin A:Acvr2 non-signaling complex into a signaling complex. The result of the Activin neo-function is that Fibro-adipogenic progenitor (FAP) cells initiate endochondral ossification. Atypical mutations involving other residues may work similarly, resulting in the ACVR1 protein to be stuck in its active conformation despite no BMP being present. Mutations in the ACVR1 gene may also be linked to diffuse intrinsic pontine glioma (DIPG).

The liver expression of the key iron regulator hepcidin is controlled by the bone morphogenic protein (BMP)/SMAD pathway. BMP signaling requires the ligand (e,g., BMP7, BMP6, or BMP2), type I receptor (e.g., ACVR1), type II receptors (e.g., ACVR2 or BMPR2), and coreceptor hemojuvelin (HJV) to phosphorylate SMAD proteins. BMP6 mediated activation of ACVR1 directly activates transcription of Hamp, the gene that encodes hepcidin. Hepcidin is a negative regulator of iron levels by causing internalization of ferroportin (slc40a1), the only known iron exporter. Inhibition of the BMP6-ACVR1 signaling cascade leads to decreased Hamp transcription, resulting in decreased circulating levels of hepcidin. A reduction of circulating hepcidin results in increased ferroportin levels, which allows increased uptake of iron from the small intestines, thereby increasing circulating iron levels.

Monoclonal antibodies to ACVR1 are described in Katagiri et al., U.S. Pat. No. 10,428,148, US Publication No. 20180118835, WO 2019172165, and in Idone et al., US Publication No. 20210253716 and WO 2021163170.

Fully human antibodies that specifically bind to ACVR1 protein, antigen-binding fragments thereof, or mutants thereof with high affinity to and that inhibit ACVR1-mediated bone morphogenetic protein (BMP) signal transduction could be important in the prevention and treatment of, e.g., heterotopic ossification, ectopic ossification, bone dysplasia, anemia, or diffuse intrinsic pontine glioma.

### BRIEF SUMMARY OF THE INVENTION

The present invention provides antibodies and antigen-binding fragments thereof that specifically bind to an Activin A receptor type 1 (ACVR1) protein and inhibit ACVR1-mediated BMP signal transduction. In certain embodiments, the anti-ACVR1 antibodies are fully human antibodies that bind to ACVR1 with high affinity and block ACVR1 or destabilize the activated conformation. The antibodies of the present invention are useful, inter alia, for deactivating or decreasing the activity of ACVR1 protein. In certain embodiments, the antibodies are useful in preventing, treating or ameliorating at least one symptom or indication of a ACVR1-associated disease or disorder in a subject. In certain embodiments, the antibodies may be administered prophylactically or therapeutically to a subject having or at risk of having a ACVR1-associated disease or disorder. In specific embodiments, the antibodies are used in the prevention and treatment of heterotopic ossification, ectopic ossification, bone dysplasia, anemia, or certain cancers, including brain tumors when administered to a subject in need thereof.

In some embodiments, the antibodies of the invention bind to an ACVR1 protein and/or a mutant thereof. Further, the antibodies disclosed herein bind to an ACVR1 protein or a mutant thereof with high affinity. ACVR1 proteins used in the present invention include ACVR1 proteins which may be derived from a mammal such as a human or a mouse. For example, the full-length amino acid sequence of human ACVR1 is available with reference to UniProtKB Accession No. Q04771 (SEQ ID NO: 61).

The ACVR1 protein may include a signal peptide occurring at positions 1-20 of ACVR1 protein, for example, of accession number Q04771 (SEQ ID NO: 61). The mature ACVR1 protein may include amino acids 21-509, for example, of accession number Q04771 (SEQ ID NO: 61). The ACVR1 protein may include an extracellular domain at amino acids 21-123 of, for example, accession number Q04771 (SEQ ID NO: 61). The ACVR1 protein may include a transmembrane domain at amino acids 124-146 of, for example, accession number Q04771 (SEQ ID NO: 61). The ACVR1 protein may include a protein kinase domain within positions 208-502, for example, of accession number Q04771 (SEQ ID NO: 61). The ACVR protein may include glycosylation at amino acid position 102 comprising an N-linked (GlcNAc . . . ) asparagine, for example, of accession number Q04771 (SEQ ID NO: 61). The ACVR protein may include a modified residue for example, such as phosphoserine at position 501, for example, of accession number Q04771 (SEQ ID NO: 61).

Mutations in the ACVR1 gene may be a responsible for various diseases including FOP. The ACVR1 protein may be a mutant ACVR1 protein having amino acid substitutions which may be found in various familial and sporadic FOP cases. The human ACVR1 protein may comprise various mutations, including but not limited to L196P (mutation that substitutes leucine at position 196 by proline), delP197_F198insL (mutation that deletes proline at position 197 and phenylalanine at position 198 and inserts leucine), R202I (mutation that substitutes arginine at position 202 by isoleucine), R206H (mutation that substitutes arginine at position 206 by histidine), Q207E (mutation that substitutes glutamine at position 207 by glutamic acid), R258S (mutation that substitutes arginine at position 258 by serine), R258G (mutation that substitutes arginine at position 258 by glycine), G325A (mutation that substitutes glycine at position 325 by alanine), G328E (mutation that substitutes glycine at position 328 by glutamic acid), G328R (mutation that substitutes glycine at position 328 by arginine), G328W (mutation that substitutes glycine at position 328 by tryptophan), G356D (mutation that substitutes glycine at position 356 by aspartic acid), and R375P (mutation that substitutes arginine at position 375 by proline) of SEQ ID NO: 61.

As another example, the full-length amino acid sequence of mouse ACVR1 protein is available with reference to Accession No. P37172 (SEQ ID NO: 62).

The antibodies of the invention can be full-length (for example, an IgG1 or IgG4 antibody) or may comprise only an antigen-binding portion (for example, a Fab, F(ab′)₂ or scFv fragment), and may be modified to affect functionality, e.g., to increase persistence in the host or to eliminate residual effector functions (Reddy et al., 2000, J. Immunol. 164:1925-1933). In certain embodiments, the antibodies may be bispecific.

In a first aspect, the present invention provides isolated recombinant monoclonal antibodies or antigen-binding fragments thereof that bind specifically to an ACVR1 protein. In some embodiments, the antibodies are fully human monoclonal antibodies.

Exemplary anti-ACVR1 antibodies of the present invention comprise the amino acid sequences and nucleic acid sequences listed in Tables 1, 2, and 3 herein. Table 1 sets forth the amino acid sequence identifiers of the heavy chain variable regions (HCVRs), light chain variable regions (LCVRs), heavy chain complementarity determining regions (HCDRs) (HCDR1, HCDR2 and HCDR3), and light chain complementarity determining regions (LCDRs) (LCDR1, LCDR2 and LCDR3) of exemplary antibodies. Table 2 sets forth the nucleic acid sequence identifiers of the HCVRs, LCVRs, HCDR1, HCDR2 HCDR3, LCDR1, LCDR2 and LCDR3 of the exemplary antibodies. Table 3 sets forth the heavy chain and light chain amino acid sequences and nucleic acid sequences of the exemplary antibodies.

The present invention provides antibodies, or antigen-binding fragments thereof, comprising an HCVR comprising an amino acid sequence selected from any of the HCVR amino acid sequences listed in Table 1, or a substantially similar sequence thereof having at least 90%, at least 95%, at least 98% or at least 99% sequence identity thereto.

The present invention also provides antibodies, or antigen-binding fragments thereof, comprising an LCVR comprising an amino acid sequence selected from any of the LCVR amino acid sequences listed in Table 1, or a substantially similar sequence thereof having at least 90%, at least 95%, at least 98% or at least 99% sequence identity thereto.

The present invention also provides antibodies, or antigen-binding fragments thereof, comprising an HCVR and an LCVR amino acid sequence pair (HCVR/LCVR) comprising any of the HCVR amino acid sequences listed in Table 1 paired with any of the LCVR amino acid sequences listed in Table 1. According to certain embodiments, the present invention provides antibodies, or antigen-binding fragments thereof, comprising an HCVR/LCVR amino acid sequence pair contained within any of the exemplary anti-ACVR1 antibodies listed in Table 1. The present invention provides antibodies, or antigen-binding fragments thereof comprising a heavy chain variable region (HCVR) having an amino acid sequence selected from the group consisting of SEQ ID NO: 14, 30, and 46, or a substantially similar sequence thereof having at least 90%, at least 95%, at least 98% or at least 99% sequence identity.

The present invention also provides an antibody or antigen-binding fragment of an antibody comprising a light chain variable region (LCVR) having an amino acid sequence selected from the group consisting of SEQ ID NO: 22, 38, and 54, or a substantially similar sequence thereof having at least 90%, at least 95%, at least 98% or at least 99% sequence identity.

The present invention also provides an antibody or antigen-binding fragment thereof comprising a HCVR and LCVR (HCVR/LCVR) sequence pair selected from the group consisting of SEQ ID NO: 14/22, 30/38, and 46/54.

The present invention also provides an antibody or antigen-binding fragment of an antibody comprising a heavy chain CDR3 (HCDR3) domain having an amino acid sequence selected from the group consisting of SEQ ID NO: 20, 36, and 52, or a substantially similar sequence thereof having at least 90%, at least 95%, at least 98% or at least 99% sequence identity; and a light chain CDR3 (LCDR3) domain having an amino acid sequence selected from the group consisting of SEQ ID NO: 28, 44, and 60, or a substantially similar sequence thereof having at least 90%, at least 95%, at least 98% or at least 99% sequence identity.

In certain embodiments, the antibody or antigen-binding portion of an antibody comprises a HCDR3/LCDR3 amino acid sequence pair selected from the group consisting of SEQ ID NO: 20/28, 36/44, and 52/60.

The present invention also provides an antibody or fragment thereof further comprising a heavy chain CDR1 (HCDR1) domain having an amino acid sequence selected from the group consisting of SEQ ID NO: 16, 32, and 48, or a substantially similar sequence thereof having at least 90%, at least 95%, at least 98% or at least 99% sequence identity; a heavy chain CDR2 (HCDR2) domain having an amino acid sequence selected from the group consisting of SEQ ID NO: 18, 34, and 50, or a substantially similar sequence thereof having at least 90%, at least 95%, at least 98% or at least 99% sequence identity; a light chain CDR1 (LCDR1) domain having an amino acid sequence selected from the group consisting of SEQ ID NO: 24, 40, and 56, or a substantially similar sequence thereof having at least 90%, at least 95%, at least 98% or at least 99% sequence identity; and a light chain CDR2 (LCDR2) domain having an amino acid sequence selected from the group consisting of SEQ ID NO: 26, 42, and 58, or a substantially similar sequence thereof having at least 90%, at least 95%, at least 98% or at least 99% sequence identity.

Certain non-limiting, exemplary antibodies and antigen-binding fragments of the invention comprise HCDR1-HCDR2-HCDR3-LCDR1-LCDR2-LCDR3 domains, respectively, having the amino acid sequences selected from the group consisting of: SEQ ID NOs: 16-18-20-24-26-28 (e.g., REGN 5166); 32-34-36-40-42-44 (REGN 5167); and 48-50-52-56-58-60 (e.g., REGN 5168).

In a related embodiment, the invention includes an antibody or antigen-binding fragment of an antibody which specifically binds Activin A receptor type 1 (ACVR1) and/or ACVR1 mutant proteins, wherein the antibody or fragment comprises the heavy and light chain CDR domains contained within heavy and light chain variable region (HCVR/LCVR) sequences selected from the group consisting of SEQ ID NO: 14/22, 30/38, and 46/54.

The present invention also provides antibodies, or antigen-binding fragments thereof, comprising a HCVR and a LCVR, said HCVR comprising an amino acid sequence listed in Table 1 having no more than twelve amino acid substitutions, and/or said LCVR comprising an amino acid sequence listed in Table 1 having no more than ten amino acid substitutions. For example, the present invention provides antibodies or antigen-binding fragments thereof comprising a HCVR and a LCVR, said HCVR comprising an amino acid sequence listed in Table 1, said amino acid sequence having one, two, three, four, five, six, seven, eight, nine, ten, eleven or twelve amino acid substitutions. In another example, the present invention provides antibodies or antigen-binding fragments thereof comprising a HCVR and a LCVR, said LCVR comprising an amino acid sequence listed in Table 1, said amino acid sequence having one, two, three, four, five, six, seven, eight, nine or ten amino acid substitutions. In one embodiment, the present invention provides anti-ACVR1 antibodies or antigen-binding fragments thereof comprising a HCVR and a LCVR, said HCVR comprising an amino acid sequence listed in Table 1, said amino acid sequence having at least one amino acid substitution, and/or said LCVR comprising an amino acid sequence listed in Table 1, said amino acid sequence having at least one amino acid substitution.

The present invention also provides antibodies, or antigen-binding fragments thereof, comprising a heavy chain CDR1 (HCDR1) comprising an amino acid sequence selected from any of the HCDR1 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a heavy chain CDR2 (HCDR2) comprising an amino acid sequence selected from any of the HCDR2 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a heavy chain CDR3 (HCDR3) comprising an amino acid sequence selected from any of the HCDR3 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a light chain CDR1 (LCDR1) comprising an amino acid sequence selected from any of the LCDR1 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a light chain CDR2 (LCDR2) comprising an amino acid sequence selected from any of the LCDR2 amino acid sequences listed in Table 1 or a substantially similar sequence thereof having at least 90%, at least 95%, at least 98% or at least 99% sequence identity.

The present invention also provides antibodies, or antigen-binding fragments thereof, comprising a light chain CDR3 (LCDR3) comprising an amino acid sequence selected from any of the LCDR3 amino acid sequences listed in Table 1 or a substantially similar sequence thereof having at least 90%, at least 95%, at least 98% or at least 99% sequence identity.

The present invention also provides antibodies, or antigen-binding fragments thereof, comprising an HCDR3 and an LCDR3 amino acid sequence pair (HCDR3/LCDR3) comprising any of the HCDR3 amino acid sequences listed in Table 1 paired with any of the LCDR3 amino acid sequences listed in Table 1.

According to certain embodiments, the present invention provides antibodies, or antigen-binding fragments thereof, comprising an HCDR3/LCDR3 amino acid sequence pair contained within any of the exemplary anti-ACVR1 antibodies listed in Table 1. In certain embodiments, the HCDR3/LCDR3 amino acid sequence pair is selected from the group consisting of SEQ ID NOs:20/28 (e.g., REGN 5166), 36/44 (e.g., REGN 5167), and 52/60 (e.g., REGN 5168).

The present invention also provides antibodies, or antigen-binding fragments thereof, comprising a HCVR and a LCVR, said HCVR comprising HCDR1 comprising an amino acid sequence differing from an amino acid sequence listed in Table 1 by 1 amino acid, HCDR2 comprising an amino acid sequence differing from an amino acid sequence listed in Table 1 by 1 amino acid, and HCDR3 comprising an amino acid sequence differing from an amino acid sequence listed in Table 1 by 1 amino acid. In certain embodiments, the present invention provides antibodies, or antigen-binding fragments thereof, comprising a HCVR and a LCVR, said LCVR comprising LCDR1 comprising an amino acid sequence differing from an amino acid sequence listed in Table 1 by 1 amino acid, LCDR2 comprising an amino acid sequence differing from an amino acid sequence listed in Table 1 by 1 amino acid, and LCDR3 comprising an amino acid sequence differing from an amino acid sequence listed in Table 1 by 1 amino acid.

For example, the present invention provides antibodies, or antigen-binding fragments thereof, comprising a HCVR and a LCVR, said HCVR comprising HCDR1 comprising an amino acid sequence of SEQ ID NO: 16, 32, or 48 or an amino acid sequence differing from SEQ ID NO: 16, 32, or 48 by 1 amino acid, HCDR2 comprising an amino acid sequence of SEQ ID NO: 18, 34, or 50 or an amino acid sequence differing from SEQ ID NO: 18, 34, or 50 by 1 amino acid, and HCDR3 comprising an amino acid sequence of SEQ ID NO: 20, 36, or 52 or an amino acid sequence differing from SEQ ID NO: 20, 36, or 52 by 1 amino acid. In another exemplary embodiment, the present invention provides antibodies, or antigen-binding fragments thereof, comprising a HCVR and a LCVR, said LCVR comprising LCDR1 comprising an amino acid sequence of SEQ ID NO: 24, 40, or 56 or an amino acid sequence differing from SEQ ID NO: 24, 40, or 56 by 1 amino acid, LCDR2 comprising an amino acid sequence of SEQ ID NO: 26, 42, or 58 or an amino acid sequence differing from SEQ ID NO: 26, 42, or 58 by 1 amino acid, and LCDR3 comprising an amino acid sequence of SEQ ID NO: 28, 44, or 60 or an amino acid sequence differing from SEQ ID NO: 28, 44, or 60 by 1 amino acid.

The present invention also provides antibodies, or antigen-binding fragments thereof, comprising a set of six CDRs HCDR1-HCDR2-HCDR3-LCDR1-LCDR2-LCDR3) contained within any of the exemplary antibodies listed in Table 1. In certain embodiments, the HCDR1-HCDR2-HCDR3-LCDR1-LCDR2-LCDR3 amino acid sequence set is selected from the group consisting of SEQ ID NOs: 16-18-20-24-26-28 (e.g., REGN 5166); 32-34-36-40-42-44 (REGN 5167); and 48-50-52-56-58-60 (e.g., REGN 5168).

In a related embodiment, the present invention provides antibodies, or antigen-binding fragments thereof, comprising a set of six CDRs HCDR1-HCDR2-HCDR3-LCDR1-LCDR2-LCDR3) contained within an HCVR/LCVR amino acid sequence pair as defined by any of the exemplary antibodies listed in Table 1. For example, the present invention includes antibodies, or antigen-binding fragments thereof, comprising the HCDR1-HCDR2-HCDR3-LCDR1-LCDR2-LCDR3 amino acid sequences set contained within an HCVR/LCVR amino acid sequence pair selected from the group consisting of SEQ ID NO: 14/22, 30/38, and 46/54.

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

In certain embodiments, the present invention includes an antibody or antigen-binding fragment thereof that binds specifically to ACVR1, wherein the antibody or antigen-binding fragment thereof comprises three heavy chain complementarity determining regions (CDRs) (HCDR1, HCDR2 and HCDR3) contained within a heavy chain variable region (HCVR) and three light chain CDRs (LCDR1, LCDR2 and LCDR3) contained within a light chain variable region (LCVR), wherein the HCVR comprises: (i) an amino acid sequence selected from the group consisting of SEQ ID NOs: 14, 30, and 46; (ii) an amino acid sequence having at least 90% identity to the amino acid sequence selected from the group consisting of SEQ ID NOs: 14, 30, and 46; (iii) an amino acid sequence having at least 95% identity to the amino acid sequence selected from the group consisting of SEQ ID NOs: 14, 30, and 46; or (iv) an amino acid sequence selected from the group consisting of SEQ ID NOs: 14, 30, and 46, said amino acid sequence having no more than 12 amino acid substitutions; and the LCVR comprises: (a) an amino acid sequence selected from the group consisting of SEQ ID NOs: 22, 38, and 54; (b) an amino acid sequence having at least 90% identity to the amino acid sequence selected from the group consisting of SEQ ID NOs: 22, 38, and 54; (c) an amino acid sequence having at least 95% identity to the amino acid sequence selected from the group consisting of SEQ ID NOs: 22, 38, and 54; or (d) an amino acid sequence selected from the group consisting of SEQ ID NOs: 22, 38, and 54, said amino acid sequence having no more than 10 amino acid substitutions.

In certain preferred embodiments, the present invention includes antibodies or antigen-binding fragments thereof that bind specifically to ACVR1 in an antagonist manner, i.e., decrease or block ACVR1 binding and/or activity.

In some embodiments, the anti-ACVR1 antibodies or antigen-binding fragments thereof of the present invention reduce trauma-induced heterotopic ossification (HO). In some cases, the anti-ACVR1 antibodies or antigen-binding fragments thereof of the present invention can reduce recurrence of trauma-induced heterotopic ossification (HO) after surgical resection.

Anti-ACVR1 antibodies or antigen-binding fragments thereof are provided for use in treating, preventing, or ameliorating at least one symptom or indication of a ACVR1-associated disease or disorder, wherein the ACVR1-associated disease or disorder is selected from the group consisting of heterotopic ossification, trauma-induced heterotopic ossification, ectopic ossification, bone dysplasia, anemia, and diffuse intrinsic pontine glioma. In some cases, the ACVR1-associated disease or disorder is not fibroplasia ossificans progressiva. Anti-ACVR1 antibodies or antigen-binding fragments thereof are provided for use in reducing recurrence of trauma-induced heterotopic ossification (HO) after surgical resection.

The present invention includes anti-ACVR1 antibodies having a modified glycosylation pattern. In some embodiments, modification to remove undesirable glycosylation sites may be useful, or an antibody lacking a fucose moiety present on the oligosaccharide chain, for example, to increase antibody dependent cellular cytotoxicity (ADCC) function (see Shield et al. (2002) JBC 277:26733). In other applications, modification of galactosylation can be made in order to modify complement dependent cytotoxicity (CDC).

In certain embodiments, the present invention provides antibodies and antigen-binding fragments thereof that exhibit pH-dependent binding to ACVR1. For example, the present invention includes antibodies and antigen-binding fragment thereof that bind ACVR1 with higher affinity at neutral pH than at acidic pH (i.e., reduced binding at acidic pH).

The present invention also provides for antibodies and antigen-binding fragments thereof that compete for specific binding to ACVR1 with an antibody or antigen-binding fragment thereof comprising the CDRs of a HCVR and the CDRs of a LCVR, wherein the HCVR and LCVR each has an amino acid sequence selected from the HCVR and LCVR sequences listed in Table 1.

The present invention also provides antibodies and antigen-binding fragments thereof that cross-compete for binding to ACVR1 with a reference antibody or antigen-binding fragment thereof comprising the CDRs of a HCVR and the CDRs of a LCVR, wherein the HCVR and LCVR each has an amino acid sequence selected from the HCVR and LCVR sequences listed in Table 1.

The present invention also provides antibodies and antigen-binding fragments thereof that bind to the same epitope as a reference antibody or antigen-binding fragment thereof comprising three CDRs of a HCVR and three CDRs of a LCVR, wherein the HCVR and LCVR each has an amino acid sequence selected from the HCVR and LCVR sequences listed in Table 1.

The present invention also provides isolated antibodies and antigen-binding fragments thereof that inhibit ligand-induced signaling by BMP7, Activin A or other TGFBeta family ligand forming a signaling complex with an Activin type II receptor. In some embodiments, the antibody or antigen-binding fragment thereof prevents ACVR1 from forming signaling complex with an Activin type II receptor. The present invention provides isolated antibodies and antigen-binding fragments thereof that may bind to the same epitope on ACVR1 as BMP7 or Activin A or an Activin type II receptor or may bind to a different epitope on ACVR1 as BMP7 or Activin A or an Activin type II receptor.

In certain embodiments, the antibodies or antigen-binding fragments of the present invention are bispecific comprising a first binding specificity to a first epitope of ACVR1 and a second binding specificity to a second epitope of ACVR1 wherein the first and second epitopes are distinct and non-overlapping.

In certain embodiments, the present invention provides an isolated antibody or antigen-binding fragment thereof that has one or more of the following characteristics:
(a) is a fully human monoclonal antibody;
(b) binds to human ACVR1 extracellular domain fused to mFc (SEQ ID NO: 64) at 37° C. with a dissociation constant (K_{D}) of less than 15 nM, less than 10 nM, less than less than 5 nM, less than 3 nM, less than 2 nM, less than 1 nM, less than 0.5 nM, less than 0.3, less than 0.2 nM, or less than 0.1 nM as measured in a surface plasmon resonance assay;
(c) binds to human ACVR1 extracellular domain fused to myc-myc-hexahistag (e.g., SEQ ID NO: 63) at 37° C. with a K_{D} of less than 50 nM, less than 10 nM, less than 5 nM, less than 3 nM, less than 2 nM, less than 1 nM less than 0.5 nM as measured in a surface plasmon resonance assay;
(d) binds to mouse ACVR1 extracellular domain fused to myc-myc-hexahistag (e.g., SEQ ID NO: 65) at 37° C. with a K_{D} of less than 50 nM, less than 10 nM, less than 5 nM, less than 3 nM, less than 2 nM, less than 1 nM less than 0.5 nM as measured in a surface plasmon resonance assay;
(e) binds to mouse ACVR1 extracellular domain fused to mFc at 37° C. with a K_{D} of less than 10 nM, less than 5 nM, less than 3 nM, less than 2 nM, less than 1 nM less than 0.5 nM, less than 0.2 nM, or less than 0.1 nM;
(f) binds to cells expressing human ACVR1 protein or human ACVR (R206H) protein;
(g) inhibits activation of cells expressing human ACVR1(R206H) by human Activin A with a IC₅₀ of with a IC₅₀ of less than 10 nM, less than 5 nM, less than 3 nM, less than 2 nM, or less than 1 nM, or less as measured in a cell-based bioassay;
(h) inhibits activation of cells expressing human ACVR1(R206H) by human BMP7 with a IC₅₀ of with a IC₅₀ of less than 10 nM, less than 5 nM, less than 3 nM, less than 2 nM, or less than 1 nM, or less as measured in a cell-based bioassay; and
(i) comprises a HCVR comprising an amino acid sequence selected from the group consisting of HCVR sequence listed in Table 1 and a LCVR comprising an amino acid sequence selected from the group consisting of LCVR sequences listed in Table 1.

In a second aspect, the present invention provides nucleic acid molecules encoding anti-ACVR1 antibodies or portions thereof. For example, the present invention provides nucleic acid molecules encoding any of the HCVR amino acid sequences listed in Table 1; in certain embodiments the nucleic acid molecule comprises a polynucleotide sequence selected from any of the HCVR nucleic acid sequences listed in Table 2, or a substantially similar sequence thereof having at least 90%, at least 95%, at least 98% or at least 99% sequence identity thereto.

The present invention also provides nucleic acid molecules encoding any of the LCVR amino acid sequences listed in Table 1; in certain embodiments the nucleic acid molecule comprises a polynucleotide sequence selected from any of the LCVR nucleic acid sequences listed in Table 2, or a substantially similar sequence thereof having at least 90%, at least 95%, at least 98% or at least 99% sequence identity thereto.

The present invention also provides nucleic acid molecules encoding any of the HCDR1 amino acid sequences listed in Table 1; in certain embodiments the nucleic acid molecule comprises a polynucleotide sequence selected from any of the HCDR1 nucleic acid sequences listed in Table 2, or a substantially similar sequence thereof having at least 90%, at least 95%, at least 98% or at least 99% sequence identity thereto.

The present invention also provides nucleic acid molecules encoding any of the HCDR2 amino acid sequences listed in Table 1; in certain embodiments the nucleic acid molecule comprises a polynucleotide sequence selected from any of the HCDR2 nucleic acid sequences listed in Table 2, or a substantially similar sequence thereof having at least 90%, at least 95%, at least 98% or at least 99% sequence identity thereto.

The present invention also provides nucleic acid molecules encoding any of the HCDR3 amino acid sequences listed in Table 1; in certain embodiments the nucleic acid molecule comprises a polynucleotide sequence selected from any of the HCDR3 nucleic acid sequences listed in Table 2, or a substantially similar sequence thereof having at least 90%, at least 95%, at least 98% or at least 99% sequence identity thereto.

The present invention also provides nucleic acid molecules encoding any of the LCDR1 amino acid sequences listed in Table 1; in certain embodiments the nucleic acid molecule comprises a polynucleotide sequence selected from any of the LCDR1 nucleic acid sequences listed in Table 2, or a substantially similar sequence thereof having at least 90%, at least 95%, at least 98% or at least 99% sequence identity thereto.

The present invention also provides nucleic acid molecules encoding any of the LCDR2 amino acid sequences listed in Table 1; in certain embodiments the nucleic acid molecule comprises a polynucleotide sequence selected from any of the LCDR2 nucleic acid sequences listed in Table 2, or a substantially similar sequence thereof having at least 90%, at least 95%, at least 98% or at least 99% sequence identity thereto.

The present invention also provides nucleic acid molecules encoding any of the LCDR3 amino acid sequences listed in Table 1; in certain embodiments the nucleic acid molecule comprises a polynucleotide sequence selected from any of the LCDR3 nucleic acid sequences listed in Table 2, or a substantially similar sequence thereof having at least 90%, at least 95%, at least 98% or at least 99% sequence identity thereto.

The present invention also provides nucleic acid molecules encoding an HCVR, wherein the HCVR comprises a set of three CDRs HCDR1-HCDR2-HCDR3), wherein the HCDR1-HCDR2-HCDR3 amino acid sequence set is as defined by any of the exemplary antibodies listed in Table 1.

The present invention also provides nucleic acid molecules encoding an LCVR, wherein the LCVR comprises a set of three CDRs LCDR1-LCDR2-LCDR3), wherein the LCDR1-LCDR2-LCDR3 amino acid sequence set is as defined by any of the exemplary antibodies listed in Table 1.

The present invention also provides nucleic acid molecules encoding both an HCVR and an LCVR, wherein the HCVR comprises an amino acid sequence of any of the HCVR amino acid sequences listed in Table 1, and wherein the LCVR comprises an amino acid sequence of any of the LCVR amino acid sequences listed in Table 1. In certain embodiments, the nucleic acid molecule comprises a polynucleotide sequence selected from any of the HCVR nucleic acid sequences listed in Table 2, or a substantially similar sequence thereof having at least 90%, at least 95%, at least 98% or at least 99% sequence identity thereto, and a polynucleotide sequence selected from any of the LCVR nucleic acid sequences listed in Table 1, or a substantially similar sequence thereof having at least 90%, at least 95%, at least 98% or at least 99% sequence identity thereto. In certain embodiments according to this aspect of the invention, the nucleic acid molecule encodes an HCVR and LCVR, wherein the HCVR and LCVR are both derived from the same anti-ACVR1 antibody listed in Table 1.

In a related aspect, the present invention provides recombinant expression vectors capable of expressing a polypeptide comprising a heavy and/or light chain variable region of an antibody. For example, the present invention includes recombinant expression vectors comprising any of the nucleic acid molecules mentioned above, i.e., nucleic acid molecules encoding any of the HCVR, LCVR, and/or CDR sequences as set forth in Table 2. In certain embodiments, the present invention provides expression vectors comprising: (a) a nucleic acid molecule comprising a nucleic acid sequence encoding a HCVR of an antibody that binds ACVR1, wherein the HCVR comprises an amino acid sequence selected from the group consisting of sequences listed in Table 1; and/or (b) a nucleic acid molecule comprising a nucleic acid sequence encoding a LCVR of an antibody that binds ACVR1, wherein the LCVR comprises an amino acid sequence selected from the group consisting of sequences listed in Table 1. Also included within the scope of the present invention are host cells into which such vectors have been introduced, as well as methods of producing the antibodies or portions thereof by culturing the host cells under conditions permitting production of the antibodies or antibody fragments, and recovering the antibodies and antibody fragments so produced. In certain embodiments, the host cells comprise a mammalian cell or a prokaryotic cell. In certain embodiments, the host cell is a Chinese Hamster Ovary (CHO) cell or an Escherichia coli (E. coli) cell. In certain embodiments, the present invention provides methods of producing an antibody or antigen-binding fragment thereof of the invention, the methods comprising introducing into a host cell an expression vector comprising a nucleic acid sequence encoding a HCVR and/or LCVR of an antibody or antigen-binding fragment thereof of the invention operably linked to a promoter; culturing the host cell under conditions favorable for expression of the nucleic acid sequence; and isolating the antibody or antigen-binding fragment thereof from the culture medium and/or host cell. The isolated antibody or antigen-binding fragment thereof may be purified using any of the methods known in prior art.

In a third aspect, the invention provides a pharmaceutical composition comprising a therapeutically effective amount of at least one recombinant monoclonal antibody or antigen-binding fragment thereof which specifically binds ACVR1 and a pharmaceutically acceptable carrier. In a related aspect, the invention features a composition which is a combination of an anti-ACVR1 antibody and a second therapeutic agent. In one embodiment, the second therapeutic agent is any agent that is advantageously combined with an anti-ACVR1 antibody.

Exemplary agents that may be advantageously combined with an anti-ACVR1 antibody include, without limitation, other agents that bind and/or activate ACVR1 activity (including other antibodies or antigen-binding fragments thereof, etc.) and/or agents which do not directly bind ACVR1 but nonetheless treat or ameliorate at least one symptom or indication of a ACVR1-associated disease or disorder (disclosed elsewhere herein). Additional combination therapies and co-formulations involving the anti-ACVR1 antibodies of the present invention are disclosed elsewhere herein.

In a fourth aspect, the invention provides therapeutic methods for treating a disease or disorder associated with ACVR1 in a subject using an anti-ACVR1 antibody or antigen-binding portion of an antibody of the invention, wherein the therapeutic methods comprise administering a therapeutically effective amount of a pharmaceutical composition comprising an antibody or antigen-binding fragment of an antibody of the invention to the subject in need thereof. The disorder treated is any disease or condition which is improved, ameliorated, inhibited or prevented by potentiation of ACVR1 activity (e.g., anemia, heterotopic ossification, ectopic ossification, bone dysplasia, or diffuse intrinsic pontine glioma). In certain embodiments, the invention provides methods to prevent, or treat a ACVR1-associated disease or disorder comprising administering a therapeutically effective amount of an anti-ACVR1 antibody or antigen-binding fragment thereof of the invention to a subject in need thereof. In some embodiments, the antibody or antigen-binding fragment thereof may be administered prophylactically or therapeutically to a subject having or at risk of having a ACVR1-associated disease or disorder. In certain embodiments, the antibody or antigen-binding fragment thereof the invention is administered in combination with a second therapeutic agent to the subject in need thereof.

The second therapeutic agent may be selected from the group consisting of an anti-Activin A antibody or antigen-binding fragment thereof, anti-BMP7 antibody or antigen binding fragment thereof, anti-ACVR2 antibody or antigen-binding fragment thereof, anti-inflammatory drugs, steroids, bisphosphonates, muscle relaxants, or retinoic acid receptor (RAR) gamma agonists, a lifestyle modification, a dietary supplement and any other drug or therapy known in the art. In certain embodiments, the second therapeutic agent may be an agent that helps to counteract or reduce any possible side effect(s) associated with an antibody or antigen-binding fragment thereof of the invention, if such side effect(s) should occur. The antibody or fragment thereof may be administered subcutaneously, intravenously, intradermally, intraperitoneally, orally, intramuscularly, or intracerebroventricularly. The antibody or fragment thereof may be administered at a dose of about 0.1 mg/kg of body weight to about 100 mg/kg of body weight of the subject. In certain embodiments, an antibody of the present invention may be administered at one or more doses comprising between 10 mg to 600 mg.

The present invention also includes use of an anti-ACVR1 antibody or antigen-binding fragment thereof of the invention in the manufacture of a medicament for the treatment of a disease or disorder that would benefit from the activation of ACVR1 binding and/or activity.

Other embodiments will become apparent from a review of the ensuing detailed description.

### BRIEF DESCRIPTION OF THE FIGURES

FIG. 1A is a matrix showing the results of an antibody cross-competition assay in which a first anti-ACVR1 antibody was applied to an hACVR1.mmh captured on a His antibody coated biosensor-tip, followed by immersion in a solution of a second anti-ACVR1 antibody (50 ug/mL). Binding response presented in white boxes indicate no competition for binding of hACVR1, suggesting distinct binding regions.

FIG. 1B shows a schematic of the antibody cross-competition assay format for which results are illustrated in FIG. 1A. A first anti-ACVR1 antibody is applied to an hACVR1.mmh captured on a His antibody coated biosensor-tip, followed by immersion in a solution of a second anti-ACVR1 antibody.

FIG. 2 shows a bar graph showing total heterotopic bone volume by micro-CT at 5 weeks and 9 weeks post-trauma in a prophylactic dosing study performed in C57BLJ6 mice receiving anti-ACVR1 antibody REGN 5168 or isotype control antibody REGN 1945. The anti-ACVR1 antibody REGN 5168 significantly attenuated post-traumatic heterotopic ossification (HO) compared to isotype control.

FIG. 3 shows a bar graph of serum hepcidin in a prophylactic dosing study performed in C57BL/6 mice receiving anti-ACVR1 antibody REGN 5168 or isotype control antibody REGN 1945. Serum hepcidin was significantly reduced in mice receiving anti-ACVR1 antibody REGN 5168 compared to control.

FIG. 4 shows a bar graph showing total heterotopic bone volume by micro-CT at 3 weeks, 6 weeks, 9 weeks, and 12-weeks post-trauma in a prophylactic dosing study performed in No MAHA transgenic mice receiving anti-ACVR1 antibodies REGN 5166, REGN 5168, or isotype control antibody REGN 1945. The anti-ACVR1 antibodies REGN 5166 and REGN 5168 each significantly attenuated post-traumatic heterotopic ossification (HO) compared to control.

FIG. 5 shows a bar graph of serum iron in a prophylactic dosing study performed in No MAHA transgenic mice receiving anti-ACVR1 antibodies REGN 5166 or REGN 5168 or isotype control antibody REGN 1945. Serum iron was significantly increased in mice receiving anti-ACVR1 antibody REGN 5168 compared to isotype control.

FIG. 6 shows a bar graph showing total heterotopic bone volume by micro-CT at 3 weeks, 6 weeks, and 9 weeks post-trauma performed in C57BL/6 mice receiving anti-ACVR1 antibody REGN 5168 or isotype control antibody REGN 1945 in a delayed dosing study with treatment starting at 3 weeks post-trauma. The anti-ACVR1 antibody REGN 5168 significantly attenuated post-traumatic heterotopic ossification (HO) at 6 weeks and 9 weeks post-trauma compared to isotype control.

FIG. 7 shows a bar graph of total heterotopic bone volume by micro-CT in No MAHA transgenic mice following HO resection surgery and commencement of treatment at 7 weeks post-trauma in the Achilles tenotomy and burn injury tHO mouse model. The arrow indicates HO resection surgery and commencement of treatment at week 7. Inhibition of ACVR1 with neutralizing antibody REGN 5168 significantly inhibited HO recurrence post-resection at week 12, week 15, and week 18 in No MAHA mice compared to mice treated with isotype control.

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, preferred methods and materials are now described. All publications, patents, and patent applications mentioned herein are incorporated herein by reference in their entirety.

### Definitions

The term “ACVR1”, also called “ALK2” refers to Activin A receptor type 1 (also known as Activin-like kinase 2). ACVR1 is a single-pass type I membrane protein. The full-length amino acid sequence of human ACVR1 is available with reference to UniProtKB Accession No. Q04771, as having 509 aa residues (SEQ ID NO: 61). The protein has an extracellular domain at amino acid residues 21-123, a transmembrane domain at amino acid positions 124-146, and a cytoplasmic domain at positions 147-509. On ligand binding, ACVR1 forms a receptor complex consisting of two type II and two type I transmembrane serine/threonine kinases. Type II receptors phosphorylate and activate type I receptors. Which autophosphorylate, then bind and activate SMAD transcriptional regulators. ACVR1 is a receptor for Activin.

The amino acid sequence of full-length human ACVR1 protein is exemplified by the amino acid sequence provided in UniProtKB/Swiss-Prot as accession number Q04771 (SEQ ID NO: 61). The full-length amino acid sequence of mouse ACVR1 protein is available with reference to Accession No. P37172 (SEQ ID NO: 62).

The term “ACVR1” includes recombinant ACVR1 protein or a fragment thereof. The term also encompasses ACVR1 protein or a fragment thereof coupled to, for example, a histidine tag, PADRE tag, mouse or human Fc, or a signal sequence (for example, SEQ ID NOs: 63-65).

The term “ACVR1” may include an ACVR1 protein or a fragment thereof comprising a mutation. For example, the mutation may be based on corresponding amino acid sequence or fragment thereof of human ACVR1 UniProtKB Accession No. Q04771, (SEQ ID NO: 61). For example, the ACVR1 protein or fragment thereof may comprise a mutation, including but not limited to L196P, delP197_F198insL, R202I, R206H, Q207E, R258S, R258G, G325A, G328E, G328R, G328W, G356D, and R375P of corresponding SEQ ID NO: 61.

The term “antibody”, as used herein, is intended to refer to immunoglobulin molecules comprised of four polypeptide chains, two heavy (H) chains and two light (L) chains inter-connected by disulfide bonds (i.e., “full antibody molecules”), as well as multimers thereof (e.g. IgM) or antigen-binding fragments thereof. Each heavy chain is comprised of a heavy chain variable region (“HCVR” or “V_{H}”) and a heavy chain constant region (comprised of domains C_{H}1, C_{H}2 and C_{H}3). Each light chain is comprised of a light chain variable region “LCVR or “V_{L}”) and a light chain constant region (C_{L}). The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In certain embodiments of the invention, the FRs of the antibody (or antigen binding fragment thereof) may be identical to the human germline sequences, or may be naturally or artificially modified. An amino acid consensus sequence may be defined based on a side-by-side analysis of two or more CDRs.

Substitution of one or more CDR residues or omission of one or more CDRs is also possible. Antibodies have been described in the scientific literature in which one or two CDRs can be dispensed with for binding. Padlan et al. (1995 FASEB J. 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et al. 2002 J Mol Biol 320:415-428).

CDR residues not contacting antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically. Empirical substitutions can be conservative or non-conservative substitutions.

The fully human anti-ACVR1 monoclonal antibodies disclosed herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The present invention includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic biological properties, reduced immunogenicity, etc. Antibodies and antigen-binding fragments obtained in this general manner are encompassed within the present invention.

The present invention also includes fully human anti-ACVR1 monoclonal antibodies comprising variants of any of the HCVR, LCVR, and/or CDR amino acid sequences disclosed herein having one or more conservative substitutions. For example, the present invention includes anti-ACVR1 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “human antibody”, or “fully human antibody”, as used herein, is intended to include antibodies having variable and constant regions derived from human germline immunoglobulin sequences. The human mAbs of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, or “fully human antibody”, as used herein, is not intended to include mAbs in which CDR sequences derived from the germline of another mammalian species (e.g., mouse), have been grafted onto human FR sequences. The term includes antibodies that are recombinantly produced in a non-human mammal, or in cells of a non-human mammal. The term is not intended to include antibodies isolated from or generated in a human subject.

The term “recombinant”, as used herein, refers to antibodies or antigen-binding fragments thereof of the invention created, expressed, isolated or obtained by technologies or methods known in the art as recombinant DNA technology which include, e.g., DNA splicing and transgenic expression. The term refers to antibodies expressed in a non-human mammal (including transgenic non-human mammals, e.g., transgenic mice), or a cell (e.g., CHO cells) expression system or isolated from a recombinant combinatorial human antibody library.

The term “specifically binds,” or “binds specifically to”, or the like, means that an antibody or antigen-binding fragment thereof forms a complex with an antigen that is relatively stable under physiologic conditions. Specific binding can be characterized by an equilibrium dissociation constant of at least about 1×10⁻⁸ M or less (e.g., a smaller K_{D} denotes a tighter binding). Methods for determining whether two molecules specifically bind are well known in the art and include, for example, equilibrium dialysis, surface plasmon resonance, and the like. As described herein, antibodies have been identified by surface plasmon resonance, e.g., BIACORE™, which bind specifically to ACVR1. Moreover, multi-specific antibodies that bind to one domain in ACVR1 and one or more additional antigens or a bi-specific that binds to two different regions of ACVR1 are nonetheless considered antibodies that “specifically bind”, as used herein.

The term “high affinity” antibody refers to those mAbs having a binding affinity to ACVR1, expressed as K_{D}, of at least 10⁻⁸ M; preferably 10⁻⁹ M; more preferably 10⁻¹⁰M, even more preferably 10⁻¹¹ M, as measured by surface plasmon resonance, e.g., BIACORE™ or solution-affinity ELISA.

By the term “slow off rate”, “Koff” or “kd” is meant an antibody that dissociates from ACVR1, with a rate constant of 1×10⁻³ s⁻¹ or less, preferably 1×10⁻⁴ s⁻¹ or less, as determined by surface plasmon resonance, e.g., BIACORE™.

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding fragment” of an antibody, or “antibody fragment”, as used herein, refers to one or more fragments of an antibody that retain the ability to bind to ACVR1 protein.

In specific embodiments, antibody or antibody fragments of the invention may be conjugated to a moiety such a ligand or a therapeutic moiety (“immunoconjugate”), a second anti-ACVR1 antibody, or any other therapeutic moiety useful for treating a ACVR1-associated disease or disorder.

An “isolated antibody”, as used herein, is intended to refer to an antibody that is substantially free of other antibodies (Abs) having different antigenic specificities (e.g., an isolated antibody that specifically binds ACVR1, or a fragment thereof, is substantially free of Abs that specifically bind antigens other than ACVR1.

An “deactivating antibody” or an “antagonist antibody”, as used herein (or an “antibody that decreases or blocks ACVR1 activity” or “an antibody that destabilizes the activated conformation”), is intended to refer to an antibody whose binding to ACVR1 results in deactivation of at least one biological activity of ACVR1. For example, an antibody of the invention may decrease anemia upon administration to a subject in need thereof.

The term “surface plasmon resonance”, as used herein, refers to an optical phenomenon that allows for the analysis of real-time biomolecular interactions by detection of alterations in protein concentrations within a biosensor matrix, for example using the BIACORE™ system (Pharmacia Biosensor AB, Uppsala, Sweden and Piscataway, N.J.).

The term “K_{D}”, as used herein, is intended to refer to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The term “cross-competes”, as used herein, means an antibody or antigen-binding fragment thereof binds to an antigen and inhibits or blocks the binding of another antibody or antigen-binding fragment thereof. The term also includes competition between two antibodies in both orientations, i.e., a first antibody that binds and blocks binding of second antibody and vice-versa. In certain embodiments, the first antibody and second antibody may bind to the same epitope. Alternatively, the first and second antibodies may bind to different, but overlapping epitopes such that binding of one inhibits or blocks the binding of the second antibody, e.g., via steric hindrance. Cross-competition between antibodies may be measured by methods known in the art, for example, by a real-time, label-free bio-layer interferometry assay. Cross-competition between two antibodies may be expressed as the binding of the second antibody that is less than the background signal due to self-self binding (wherein first and second antibodies is the same antibody). Cross-competition between 2 antibodies may be expressed, for example, as % binding of the second antibody that is less than the baseline self-self background binding (wherein first and second antibodies is the same antibody).

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45, herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215: 403-410 and (1997) Nucleic Acids Res. 25:3389-3402, each of which is herein incorporated by reference.

By the phrase “therapeutically effective amount” is meant an amount that produces the desired effect for which it is administered. The exact amount will depend on the purpose of the treatment, and will be ascertainable by one skilled in the art using known techniques (see, for example, Lloyd (1999) The Art, Science and Technology of Pharmaceutical Compounding).

As used herein, the term “subject” refers to an animal, preferably a mammal, more preferably a human, in need of amelioration, prevention and/or treatment of a ACVR1-associated disease or disorder such as anemia or ectopic ossification. The term includes human subjects who have or are at risk of having such a disease or disorder.

As used herein, the terms “treat”, “treating”, or “treatment” refer to the reduction or amelioration of the severity of at least one symptom or indication of a ACVR1-associated disease or disorder due to the administration of a therapeutic agent such as an antibody of the present invention to a subject in need thereof. The terms include inhibition of progression of disease or of worsening of a symptom/indication. The terms also include positive prognosis of disease, i.e., the subject may be free of disease or may have reduced disease upon administration of a therapeutic agent such as an antibody of the present invention. The therapeutic agent may be administered at a therapeutic dose to the subject.

The terms “prevent”, “preventing” or “prevention” refer to inhibition of manifestation of a ACVR1-associated disease or disorder or any symptoms or indications of such a disease or disorder upon administration of an antibody of the present invention.

The term “heterotopic ossification” (HO) refers to formation of benign mature bony elements in extra-skeletal sites, including soft tissue and joints.

### Antigen-Binding Fragments of Antibodies

Unless specifically indicated otherwise, the term “antibody,” as used herein, shall be understood to encompass antibody molecules comprising two immunoglobulin heavy chains and two immunoglobulin light chains (i.e., “full antibody molecules”) as well as antigen-binding fragments thereof. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding fragment” of an antibody, or “antibody fragment”, as used herein, refers to one or more fragments of an antibody that retain the ability to specifically bind to an ACVR1 protein, a fragment thereof, and/or mutant thereof. An antibody fragment may include a Fab fragment, a F(ab′)₂ fragment, a Fv fragment, a dAb fragment, a fragment containing a CDR, or an isolated CDR. In certain embodiments, the term “antigen-binding fragment” refers to a polypeptide fragment of a multi-specific antigen-binding molecule. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and (optionally) constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMI Ps), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (V) V_{H}-C_{H}1-C_{H}2-C_{H}3; V_{H}-C_{H}2-C_{H}3; (Vii) V_{H}-C_{L}; (Viii) V_{L}-C_{H}1; (ix) V_{L}-C_{H}2; (X) V_{L}-C_{H}3; (xi) V_{L}-C_{H}1-C_{H}2; (XII) V_{L}-C_{H}1-C_{H}2-C_{H}3; (Xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be mono-specific or multi-specific (e.g., bi-specific). A multi-specific antigen-binding fragment of an antibody will typically comprise at least two different variable domains, wherein each variable domain is capable of specifically binding to a separate antigen or to a different epitope on the same antigen. Any multi-specific antibody format, including the exemplary bi-specific antibody formats disclosed herein, may be adapted for use in the context of an antigen-binding fragment of an antibody of the present invention using routine techniques available in the art.

### Preparation of Human Antibodies

Methods for generating human antibodies in transgenic mice are known in the art. Any such known methods can be used in the context of the present invention to make human antibodies that specifically bind to ACVR1.

Human antibodies against ACVR1 may be isolated from a full length human IgG synthetic naive library using an in vitro yeast selection system and associated methods (Rouha H, et al. MAbs. 2015;7(1):243-254). An antibody library of approximately 1×10¹⁰ in diversity may be designed and propagated as described previously (Rouha et al. 2015, Xu et al., Protein Eng Des Sel. 2013; 26(10):663-670). ACVR1-binding antibodies may be enriched by incubation of biotinylated ACVR1-Fc and Myc-His monomeric ACVR1 at different concentrations with antibody-expressing yeast cells followed by magnetic bead selection (Miltenyi Biotec) or flow cytometry on a FACSAria II cell sorter (BD Biosciences), for example, using fluorescent streptavidin or extravidin secondary reagents in several successive selection rounds. Antibodies cross-reactive to off-target proteins ALK1, ALK3, and ALK6 may be actively depleted from selection outputs. After the last round of enrichment, yeast cells may be plated onto agar plates, analyzed by DNA sequencing, and expanded for IgG production. Heavy chains from the naive outputs may be used to prepare light-chain diversification libraries, which may then used for additional selection rounds. In particular, heavy chains may be extracted from naive selection round outputs and transformed into a light-chain library, for example, consisting of 1×10⁶ unique light chains to create new libraries of, for example, approximately 1×10⁸ in total diversity. Antibody optimization may be completed in 3 phases. Optimization of the heavy chain via diversification of the complementarity-determining regions (CDRs) HCDR1 and HCDR2 may be followed either by mutagenic PCR-based diversification of the entire heavy chain variable region or diversification of the light chain LCDR1 and LCDR2 segments. HCDR1 and HCDR2 regions may be diversified with premade libraries of HCDR1 and HCDR2 variants of a diversity of, for example, approximately 1×10⁸. Lead variants may be further diversified via DNA oligonucleotide sequence variegation of the HCDR3 or LCDR3. Diversified antibody lineage populations may be selected for enhanced binding to the target proteins while avoiding undesired cross-reactivity. The methods used for selections on diversified populations may be similar or identical to those used to isolate the original lead IgGs (Xu et al., 2013).

Alternatively, immunogen comprising any one of the following can be used to generate antibodies to ACVR1 protein. In certain embodiments, the antibodies of the invention are obtained from mice immunized with a full length, native ACVR1 protein (See, for example, UniProtKB/Swiss-Prot accession number Q04771) (SEQ ID NO: 61) or with DNA encoding the protein or fragment thereof. Alternatively, the protein or a fragment thereof may be produced using standard biochemical techniques and modified and used as immunogen.

In some embodiments, the immunogen may be a recombinant ACVR1 protein or fragment thereof expressed in E. coli or in any other eukaryotic or mammalian cells such as Chinese hamster ovary (CHO) cells (for example, SEQ ID NOs: 63-65).

Using VELOCIMMUNE® technology (see, for example, U.S. Pat. No. 6,596,541, Regeneron Pharmaceuticals, VELOCIMMUNE®) or any other known method for generating monoclonal antibodies, high affinity chimeric antibodies to ACVR1 are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. As in the experimental section below, the antibodies are characterized and selected for desirable characteristics, including affinity, selectivity, epitope, etc. The mouse constant regions are replaced with a desired human constant region to generate the fully human antibody of the invention, for example wild type or modified IgG1 or IgG4. While the constant region selected may vary according to specific use, high affinity antigen-binding and target specificity characteristics reside in the variable region.

### Bioequivalents

The anti-ACVR1 antibodies and antibody fragments of the present invention encompass proteins having amino acid sequences that vary from those of the described antibodies, but that retain the ability to bind ACVR1 protein. Such variant antibodies and antibody fragments comprise one or more additions, deletions, or substitutions of amino acids when compared to parent sequence, but exhibit biological activity that is essentially equivalent to that of the described antibodies. Likewise, the antibody-encoding DNA sequences of the present invention encompass sequences that comprise one or more additions, deletions, or substitutions of nucleotides when compared to the disclosed sequence, but that encode an antibody or antibody fragment that is essentially bioequivalent to an antibody or antibody fragment of the invention.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single dose or multiple doses. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, or potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and/or in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of the antibodies of the invention may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include antibody variants comprising amino acid changes, which modify the glycosylation characteristics of the antibodies, e.g., mutations that eliminate or remove glycosylation.

### Anti-ACVR1 Antibodies Comprising Fc Variants

According to certain embodiments of the present invention, anti-ACVR1 antibodies are provided comprising an Fc domain comprising one or more mutations which enhance or diminish antibody binding to the FcRn receptor, e.g., at acidic pH as compared to neutral pH. For example, the present invention includes anti-ACVR1 antibodies comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., A, W, H, F or Y [N434A, N434W, N434H, N434F or N434Y]); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 259I (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P). In yet another embodiment, the modification comprises a 265A (e.g., D265A) and/or a 297A (e.g., N297A) modification.

For example, the present invention includes anti-ACVR1 antibodies comprising an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); 257I and 311I (e.g., P257I and Q311I); 257I and 434H (e.g., P257I and N434H); 376V and 434H (e.g., D376V and N434H); 307A, 380A and 434A (e.g., T307A, E380A and N434A); and 433K and 434F (e.g., H433K and N434F). All possible combinations of the foregoing Fc domain mutations and other mutations within the antibody variable domains disclosed herein, are contemplated within the scope of the present invention.

The present invention also includes anti-ACVR1 antibodies comprising a chimeric heavy chain constant (C_{H}) region, wherein the chimeric C_{H} region comprises segments derived from the C_{H} regions of more than one immunoglobulin isotype. For example, the antibodies of the invention may comprise a chimeric C_{H} region comprising part or all of a C_{H}2 domain derived from a human IgG1, human IgG2 or human IgG4 molecule, combined with part or all of a C_{H}3 domain derived from a human IgG1, human IgG2 or human IgG4 molecule. According to certain embodiments, the antibodies of the invention comprise a chimeric C_{H} region having a chimeric hinge region. For example, a chimeric hinge may comprise an “upper hinge” amino acid sequence (amino acid residues from positions 216 to 227 according to EU numbering) derived from a human IgG1, a human IgG2 or a human IgG4 hinge region, combined with a “lower hinge” sequence (amino acid residues from positions 228 to 236 according to EU numbering) derived from a human IgG1, a human IgG2 or a human IgG4 hinge region. According to certain embodiments, the chimeric hinge region comprises amino acid residues derived from a human IgG1 or a human IgG4 upper hinge and amino acid residues derived from a human IgG2 lower hinge. An antibody comprising a chimeric C_{H} region as described herein may, in certain embodiments, exhibit modified Fc effector functions without adversely affecting the therapeutic or pharmacokinetic properties of the antibody. (See, e.g., U.S. Patent Application Publication 2014/0243504, the disclosure of which is hereby incorporated by reference in its entirety).

### Biological Characteristics of the Antibodies

In general, the antibodies of the present invention function by binding to ACVR1 protein and decreasing its activity. For example, the present invention includes antibodies and antigen-binding fragments of antibodies that bind human ACVR1 protein (e.g., at 25° C. or at 37° C.) with a K_{D} of less than 50 nM as measured by surface plasmon resonance, e.g., using the assay format as defined in Example 3 herein.

In certain embodiments, the antibodies or antigen-binding fragments thereof bind ACVR1 with a K_{D} of less than about 50 nM, less than about 40 nM, less than about 30 nM, less than about 20 nM, less than about 10 nM, less than about 5 nM, less than about 2.5 nM, less than about 1 nM, less than about 0.5 nM, less than about 0.3 nM, less than about 0.2 nM, less than about 0.1 nM as measured by surface plasmon resonance, e.g., using the assay format as defined in Example 3 herein, or a substantially similar assay. In certain embodiments, the present invention provides an isolated anti-ACVR1 antibody or antigen-binding fragment thereof that is a fully human monoclonal antibody.

In certain embodiments, the antibodies or antigen-binding fragments thereof bind to human ACVR1 extracellular domain fused to mFc (SEQ ID NO: 64) at 37° C. with a dissociation constant (K_{D}) of less than 15 nM, less than 10 nM, less than less than 5 nM, less than 3 nM, less than 2 nM, less than 1 nM, less than 0.5 nM, less than 0.3, less than 0.2 nM, or less than 0.1 nM as measured in a surface plasmon resonance assay, e.g., using the assay format as defined in Example 3 herein, or a substantially similar assay.

In certain embodiments, the antibodies or antigen-binding fragments thereof bind to human ACVR1 extracellular domain fused to myc-myc-hexahistag (mmh) (e.g., SEQ ID NO: 63) at 37° C. with a K_{D} of less than 50 nM, less than 10 nM, less than 5 nM, less than 3 nM, less than 2 nM, less than 1 nM less than 0.5 nM as measured in a surface plasmon resonance assay, e.g., using the assay format as defined in Example 3 herein, or a substantially similar assay.

In certain embodiments, the antibodies or antigen-binding fragments thereof bind human and mouse ACVR1, e.g., using the assay format as defined in Example 3 herein, or a substantially similar assay.

In certain embodiments, the antibodies or antigen-binding fragments thereof bind to mouse ACVR1 extracellular domain fused to myc-myc-hexahistag (mmh) (e.g., SEQ ID NO: 65) at 37° C. with a K_{D} of less than 50 nM, less than 10 nM, less than 5 nM, less than 3 nM, less than 2 nM, less than 1 nM less than 0.5 nM as measured in a surface plasmon resonance assay, e.g., using the assay format as defined in Example 3 herein, or a substantially similar assay.

In certain embodiments, the antibodies or antigen-binding fragments thereof bind to mouse ACVR1 extracellular domain fused to mFc at 37° C. with a K_{D} of less than 10 nM, less than 5 nM, less than 3 nM, less than 2 nM, less than 1 nM less than 0.5 nM, less than 0.2 nM, or less than 0.1 nM as measured in a surface plasmon resonance assay, e.g., using the assay format as defined in Example 3 herein, or a substantially similar assay.

The present invention also includes antibodies or antigen-binding fragments thereof bind to cells expressing human ACVR1 protein or human ACVR (R206H) protein, e.g., using the assay format as defined in Example 4 herein, or a substantially similar assay.

In certain embodiments, the antibodies or antigen-binding fragments thereof inhibit activation of cells expressing human ACVR1(R206H) by human Activin A with a IC₅₀ of less than 10 nM, less than 5 nM, less than 3 nM, or less than 1 nM as measured in a cell-based bioassay, e.g., using the assay format as defined in Example 5 herein, or a substantially similar assay.

In certain embodiments, the antibodies or antigen-binding fragments thereof inhibit activation of cells expressing human ACVR1(R206H) by human BMP7 with a IC₅₀ of less than 10 nM, less than 5 nM, less than 3 nM, less than 2 nM, or less than 1 nM, or less than as measured in a cell-based bioassay, e.g., using the assay format as defined in Example 5 herein, or a substantially similar assay.

The invention also includes antibodies or antigen-binding fragments thereof decrease serum hepcidin.

In certain embodiments, the antibodies or antigen-binding fragments thereof increase serum iron levels.

In certain embodiments, the antibodies or antigen-binding fragments thereof inhibit wild-type ACVR1 signaling.

In certain embodiments, the anti-ACVR antibodies or antigen-binding fragments thereof according to the invention significantly attenuate post-traumatic heterotopic ossification (HO).

In certain embodiments, the antibodies or antigen-binding fragments thereof specifically bind human ACVR1, a fragment thereof, or a mutant thereof, and comprise a HCVR comprising an amino acid sequence selected from the group consisting of HCVR sequence listed in Table 1 and a LCVR comprising an amino acid sequence selected from the group consisting of LCVR sequences listed in Table 1.

In one embodiment, the present invention provides an isolated recombinant antibody or antigen-binding fragment thereof that binds specifically to ACVR1 protein and inhibit ACVR1-mediated bone morphogenetic protein (BMP) signal transduction, wherein the antibody or fragment thereof exhibits one or more of the following characteristics:

In certain embodiments, the present invention provides an isolated antibody or antigen-binding fragment thereof that has one or more of the following characteristics: (a) is a fully human monoclonal antibody; (b) binds to human ACVR1 extracellular domain fused to mFc (SEQ ID NO: 64) at 37° C. with a dissociation constant (K_{D}) of less than 15 nM, less than 10 nM, less than less than 5 nM, less than 3 nM, less than 2 nM, less than 1 nM, less than 0.5 nM, less than 0.3, less than 0.2 nM, or less than 0.1 nM as measured in a surface plasmon resonance assay; (c) binds to human ACVR1 extracellular domain fused to myc-myc-hexahistag (e.g., SEQ ID NO: 63) at 37° C. with a K_{D} of less than 50 nM, less than 10 nM, less than 5 nM, less than 3 nM, less than 2 nM, less than 1 nM less than 0.5 nM as measured in a surface plasmon resonance assay; (d) binds to mouse ACVR1 extracellular domain fused to myc-myc-hexahistag (e.g., SEQ ID NO: 65) at 37° C. with a K_{D} of less than 50 nM, less than 10 nM, less than 5 nM, less than 3 nM, less than 2 nM, less than 1 nM less than 0.5 nM as measured in a surface plasmon resonance assay; (e) binds to mouse ACVR1 extracellular domain fused to mFc at 37° C. with a K_{D} of less than 10 nM, less than 5 nM, less than 3 nM, less than 2 nM, less than 1 nM less than 0.5 nM, less than 0.2 nM, or less than 0.1 nM; (f) binds to cells expressing human ACVR1 protein or human ACVR (R206H) protein; (g) inhibits activation of cells expressing human ACVR1(R206H) by human Activin A with a IC₅₀ of with a IC₅₀ of less than 10 nM, less than 5 nM, less than 3 nM, less than 2 nM, or less than 1 nM, or less as measured in a cell-based bioassay; (h) inhibits activation of cells expressing human ACVR1(R206H) by human BMP7 with a IC₅₀ of with a IC₅₀ of less than 10 nM, less than 5 nM, less than 3 nM, less than 2 nM, or less than 1 nM, or less as measured in a cell-based bioassay; and (i) comprises a HCVR comprising an amino acid sequence selected from the group consisting of HCVR sequence listed in Table 1 and a LCVR comprising an amino acid sequence selected from the group consisting of LCVR sequences listed in Table 1.

The antibodies of the present invention may possess one or more of the aforementioned biological characteristics, or any combinations thereof. Other biological characteristics of the antibodies of the present invention will be evident to a person of ordinary skill in the art from a review of the present disclosure including the working Examples herein.

### Epitope Mapping and Related Technologies

The present invention includes anti-ACVR1 antibodies which interact with one or more amino acids found within one or more regions of the ACVR1 protein molecule. The epitope to which the antibodies bind may consist of a single contiguous sequence of 3 or more (e.g., 3, 4, 5, 6, 7, 8, 9, 10, 11, 12, 13, 14, 15, 16, 17, 18, 19, 20 or more) amino acids located within any of the aforementioned domains of the ACVR1 protein molecule (e.g. a linear epitope in a domain). Alternatively, the epitope may consist of a plurality of non-contiguous amino acids (or amino acid sequences) located within either or both of the aforementioned domains of the protein molecule (e.g. a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, NY). Other methods include alanine scanning mutational analysis, peptide blot analysis (Reineke (2004) Methods Mol. Biol. 248: 443-63), peptide cleavage analysis crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267: 252-259; Engen and Smith (2001) Anal. Chem. 73: 256A-265A.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (see US 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the antibodies of the invention into groups of antibodies binding different epitopes.

In certain embodiments, the present invention includes anti-ACVR1 antibodies and antigen-binding fragments thereof that interact with one or more epitopes found within the extracellular domain of ACVR1. The epitope(s) may consist of one or more contiguous sequences of 3 or more (e.g., 3, 4, 5, 6, 7, 8, 9, 10, 11, 12, 13, 14, 15, 16, 17, 18, 19, 20 or more) amino acids located within the extracellular domain of ACVR1. Alternatively, the epitope may consist of a plurality of non-contiguous amino acids (or amino acid sequences) located within ACVR1 protein.

The present invention includes anti-ACVR1 antibodies that bind to the same epitope, or a portion of the epitope, as any of the specific exemplary antibodies listed in Table 1. Likewise, the present invention also includes anti-ACVR1 antibodies that compete for binding to ACVR1 protein or a fragment thereof with any of the specific exemplary antibodies listed in Table 1. For example, the present invention includes anti-ACVR1 antibodies that cross-compete for binding to ACV protein with one or more antibodies listed in Table 1.

One can easily determine whether an antibody binds to the same epitope as, or competes for binding with, a reference anti-ACVR1 antibody by using routine methods known in the art. For example, to determine if a test antibody binds to the same epitope as a reference anti-ACVR1 antibody of the invention, the reference antibody is allowed to bind to a ACVR1 protein or peptide under saturating conditions. Next, the ability of a test antibody to bind to the ACVR1 protein molecule is assessed. If the test antibody is able to bind to ACVR1 following saturation binding with the reference anti-ACVR1 antibody, it can be concluded that the test antibody binds to a different epitope than the reference anti-ACVR1 antibody. On the other hand, if the test antibody is not able to bind to the ACVR1 protein following saturation binding with the reference anti-ACVR1 antibody, then the test antibody may bind to the same epitope as the epitope bound by the reference anti-ACVR1 antibody of the invention.

To determine if an antibody competes for binding with a reference anti-ACVR1 antibody, the above-described binding methodology is performed in two orientations: In a first orientation, the reference antibody is allowed to bind to a ACVR1 protein under saturating conditions followed by assessment of binding of the test antibody to the ACVR1 molecule. In a second orientation, the test antibody is allowed to bind to a ACVR1 molecule under saturating conditions followed by assessment of binding of the reference antibody to the ACVR1 molecule. If, in both orientations, only the first (saturating) antibody is capable of binding to the ACVR1 molecule, then it is concluded that the test antibody and the reference antibody compete for binding to ACVR1. As will be appreciated by a person of ordinary skill in the art, an antibody that competes for binding with a reference antibody may not necessarily bind to the identical epitope as the reference antibody, but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

### Immunoconjugates

The invention encompasses a human anti-ACVR1 monoclonal antibody or antigen-binding fragment thereof conjugated to a therapeutic moiety (“immunoconjugate”), to treat an ACVR1-associated disease or disorder (e.g., heterotopic ossification, anemia, or ectopic ossification). As used herein, the term “immunoconjugate” refers to an antibody which is chemically or biologically linked to a radioactive agent, a cytokine, an interferon, a target or reporter moiety, an enzyme, a peptide or protein or a therapeutic agent. The antibody may be linked to the radioactive agent, cytokine, interferon, target or reporter moiety, enzyme, peptide or therapeutic agent at any location along the molecule so long as it is able to bind its target. Examples of immunoconjugates include antibody drug conjugates and antibody-toxin fusion proteins. In one embodiment, the agent may be a second different antibody to ACVR1 protein. The type of therapeutic moiety that may be conjugated to the anti-ACVR1 antibody and will take into account the condition to be treated and the desired therapeutic effect to be achieved. Examples of suitable agents for forming immunoconjugates are known in the art; see for example, WO 05/103081, which is herein incorporated by reference.

### Multi-Specific Antibodies

The antibodies of the present invention may be mono-specific, bi-specific, or multi-specific. Multi-specific antibodies may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244, each of which is herein incorporated by reference.

Any of the multi-specific antigen-binding molecules of the invention, or variants thereof, may be constructed using standard molecular biological techniques (e.g., recombinant DNA and protein expression technology), as will be known to a person of ordinary skill in the art.

In some embodiments, ACVR1-specific antibodies are generated in a bi-specific format (a “bi-specific”) in which variable regions binding to distinct domains of ACVR1 protein are linked together to confer dual-domain specificity within a single binding molecule. Appropriately designed bi-specifics may enhance overall ACVR1-protein inhibitory efficacy through increasing both specificity and binding avidity. Variable regions with specificity for individual domains, (e.g., segments of the N-terminal domain), or that can bind to different regions within one domain, are paired on a structural scaffold that allows each region to bind simultaneously to the separate epitopes, or to different regions within one domain. In one example for a bi-specific, heavy chain variable regions (V_{H}) from a binder with specificity for one domain are recombined with light chain variable regions (V_{L}) from a series of binders with specificity for a second domain to identify non-cognate V_{L} partners that can be paired with an original V_{H} without disrupting the original specificity for that V_{H}. In this way, a single V_{L} segment (e.g., V_{L}1) can be combined with two different V_{H} domains (e.g., V_{H}1 and V_{H}2) to generate a bi-specific comprised of two binding “arms” (V_{H}1-V_{L}1 and V_{H}2- V_{L}1). Use of a single V_{L} segment reduces the complexity of the system and thereby simplifies and increases efficiency in cloning, expression, and purification processes used to generate the bi-specific (See, for example, US2011/0195454 and US2010/0331527).

Alternatively, antibodies that bind more than one domains and a second target, such as, but not limited to, for example, a second different anti-ACVR1 antibody, may be prepared in a bi-specific format using techniques described herein, or other techniques known to those skilled in the art. Antibody variable regions binding to distinct regions may be linked together with variable regions that bind to relevant sites on, for example, the extracellular domain of ACVR1, to confer dual-antigen specificity within a single binding molecule. Appropriately designed bi-specifics of this nature serve a dual function. Variable regions with specificity for the extracellular domain are combined with a variable region with specificity for outside the extracellular domain and are paired on a structural scaffold that allows each variable region to bind to the separate antigens.

An exemplary bi-specific antibody format that can be used in the context of the present invention involves the use of a first immunoglobulin (Ig) C_{H}3 domain and a second Ig C_{H}3 domain, wherein the first and second Ig C_{H}3 domains differ from one another by at least one amino acid, and wherein at least one amino acid difference reduces binding of the bi-specific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). Further modifications that may be found within the second C_{H}3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies. Variations on the bi-specific antibody format described above are contemplated within the scope of the present invention.

Other exemplary bispecific formats that can be used in the context of the present invention include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mabe bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

### Therapeutic Administration and Formulations

The invention provides therapeutic compositions comprising the anti-ACVR1 antibodies or antigen-binding fragments thereof of the present invention. Therapeutic compositions in accordance with the invention will be administered with suitable pharmaceutically acceptable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antibody may vary depending upon the age and the size of a subject to be administered, target disease, conditions, route of administration, and the like. When an antibody of the present invention is used for treating a disease or disorder in an adult patient, or for preventing such a disease, it is advantageous to administer the antibody of the present invention normally at a single dose of about 0.1 to about 100 mg/kg body weight. Depending on the severity of the condition, the frequency and the duration of the treatment can be adjusted. In certain embodiments, the antibody or antigen-binding fragment thereof of the invention can be administered as an initial dose of at least about 0.1 mg to about 800 mg, about 1 to about 600 mg, about 5 to about 500 mg, or about 10 to about 400 mg. In certain embodiments, the initial dose may be followed by administration of a second or a plurality of subsequent doses of the antibody or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al. (1987) J. Biol. Chem. 262:4429-4432). Methods of introduction include, but are not limited to, intradermal, transdermal, intramuscular, intraperitoneal, intravenous, subcutaneous, intranasal, epidural, intracerebroventricular, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. The pharmaceutical composition can be also delivered in a vesicle, in particular a liposome (see, for example, Langer (1990) Science 249:1527-1533).

The use of nanoparticles to deliver the antibodies of the present invention is also contemplated herein. Antibody-conjugated nanoparticles may be used both for therapeutic and diagnostic applications. Antibody-conjugated nanoparticles and methods of preparation and use are described in detail by Arruebo, M., et al. 2009 (“Antibody-conjugated nanoparticles for biomedical applications” in J. Nanomat. Volume 2009, Article ID 439389, 24 pages, doi: 10.1155/2009/439389), incorporated herein by reference. Nanoparticles may be developed and conjugated to antibodies contained in pharmaceutical compositions to target cells. Nanoparticles for drug delivery have also been described in, for example, U.S. Pat. Nos. 8,257,740, or 8,246,995, each incorporated herein in its entirety.

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracranial, intraperitoneal and intramuscular injections, drip infusions, etc. These injectable preparations may be prepared by methods publicly known.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In treatment of certain diseases or conditions (e.g., DI PG), it may be necessary to overcome the blood-brain barrier. In certain embodiments, the blood-brain barrier is overcome by using one or more approaches disclosed in the art, e.g., in Parodi et al., 2019, Pharmaceutics 11:245, which is herein incorporated by reference.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the antibody contained is generally about 5 to about 500 mg per dosage form in a unit dose; especially in the form of injection, it is preferred that the antibody is contained in about 5 to about 300 mg and in about 10 to about 300 mg for the other dosage forms.

### Therapeutic Uses of the Antibodies

The antibodies or antigen-binding fragments thereof of the present invention are useful for the treatment, and/or prevention of a disease or disorder or condition associated with ACVR1 and/or for ameliorating at least one symptom associated with such disease, disorder or condition. In certain embodiments, an antibody or antigen-binding fragment thereof of the invention may be administered at a therapeutic dose to a patient with a disease or disorder or condition associated with ACVR1 or a mutant ACVR protein.

In certain embodiments, the antibodies or antigen-binding fragments thereof of the present invention are useful for treating or preventing at least one symptom or indication of an ACVR1-associated or ACVR1 mutant protein-associated disease or disorder selected from the group consisting of heterotopic ossification, trauma-induced heterotopic ossification, ectopic ossification, bone dysplasia, anemia, and diffuse intrinsic pontine glioma. In some cases, the ACVR1-associated or ACVR1 mutant protein-associated disease or disorder is not fibroplasia ossificans progressiva (FOP). In some cases, the ACVR1 mutant protein-associated disease or disorder is FOP.

It is also contemplated herein to use one or more antibodies of the present invention prophylactically to subjects at risk for suffering from a ACVR1-associated disease or disorder.

In one embodiment of the invention, the present antibodies are used for the preparation of a pharmaceutical composition or medicament for treating patients suffering from a disease, disorder or condition disclosed herein. In another embodiment of the invention, the present antibodies are used as adjunct therapy with any other agent or any other therapy known to those skilled in the art useful for treating or ameliorating a disease, disorder or condition disclosed herein.

### Combination Therapies

Combination therapies may include an antibody of the invention and any additional therapeutic agent that may be advantageously combined with an antibody of the invention, or with a biologically active fragment of an antibody of the invention. The antibodies of the present invention may be combined synergistically with one or more drugs or therapy used to treat an ACVR1-associated or ACVR1 mutant protein-associated disease or disorder. In some embodiments, the antibodies of the invention may be combined with a second therapeutic agent to ameliorate one or more symptoms of said disease or condition.

Depending upon the disease, disorder or condition, the antibodies of the present invention may be used in combination with one or more additional therapeutic agents.

Examples of the additional therapeutic drug for ectopic ossification that can be administered in combination with the anti-ACVR1 antibody can include, but are not limited to, anti-Activin A inhibitor or antigen binding fragment thereof, and an anti-ACVR2 antibody or antigen-binding fragment thereof, anti-inflammatory drugs, steroids, bisphosphonates, muscle relaxants, and retinoic acid receptor (RAR) gamma agonists.

Activins belong to the transforming growth factor-beta (TGF-β) superfamily and exert a broad range of biological effects on cell proliferation, differentiation, metabolism, homeostasis, and apoptosis, as well as immune response and tissue repair. Activin A is a disulfide-linked homodimer (two beta-A chains) that binds to and activates heteromeric complexes of a type I (Act RI-A and Act RI-B) and a type II (Act RII-A and Act RII-B) serine-threonine kinase receptor. Activin A may act as a ligand to ACVR1 proteins or ACVR1 mutant proteins.

Examples of the anti-inflammatory drug can include aspirin, diclofenac, indomethacin, ibuprofen, ketoprofen, naproxen, piroxicam, rofecoxib, celecoxib, azathioprine, penicillamine, methotrexate, sulfasalazine, leflunomide, infliximab, and etanercept. Examples of the steroid can include prednisolone, beclomethasone, betamethasone, fluticasone, dexamethasone, and hydrocortisone. Examples of the bisphosphonate can include alendronate, cimadronate, clodronate, etidronate, ibandronate, incadronate, minodronate, neridronate, olpadronate, pamidronate, piridronate, risedronate, tiludronate, and zoledronate. Examples of the muscle relaxant can include cyclobenzaprine, metaxalone, and baclofen. Examples of the retinoic acid receptor gamma agonist can include palovarotene. Examples of the additional therapeutic drug for anemia may include recombinant erythropoietin (EPO) and iron supplements. Examples of additional therapeutic treatments for diffuse intrinsic pontine glioma may include radiation therapy, or experimental chemotherapy.

As used herein, the term “in combination with” means that additional therapeutically active component(s) may be administered prior to, concurrent with, or after the administration of the anti-ACVR1 antibody of the present invention. The term “in combination with” also includes sequential or concomitant administration of an anti-ACVR1 antibody and a second therapeutic agent.

The additional therapeutically active component(s) may be administered to a subject prior to administration of an anti-ACVR1 antibody of the present invention. For example, a first component may be deemed to be administered “prior to” a second component if the first component is administered 1 week before, 72 hours before, 60 hours before, 48 hours before, 36 hours before, 24 hours before, 12 hours before, 6 hours before, 5 hours before, 4 hours before, 3 hours before, 2 hours before, 1 hour before, 30 minutes before, or less than 30 minutes before administration of the second component. In other embodiments, the additional therapeutically active component(s) may be administered to a subject after administration of an anti-ACVR1 antibody of the present invention. For example, a first component may be deemed to be administered “after” a second component if the first component is administered 30 minutes after, 1 hour after, 2 hours after, 3 hours after, 4 hours after, 5 hours after, 6 hours after, 12 hours after, 24 hours after, 36 hours after, 48 hours after, 60 hours after, 72 hours after or more after administration of the second component. In yet other embodiments, the additional therapeutically active component(s) may be administered to a subject concurrent with administration of an anti-ACVR1 antibody of the present invention. “Concurrent” administration, for purposes of the present invention, includes, e.g., administration of an anti-ACVR1 antibody and an additional therapeutically active component to a subject in a single dosage form, or in separate dosage forms administered to the subject within about 30 minutes or less of each other. If administered in separate dosage forms, each dosage form may be administered via the same route (e.g., both the anti-ACVR1 antibody and the additional therapeutically active component may be administered intravenously, etc.); alternatively, each dosage form may be administered via a different route (e.g., the anti-ACVR1 antibody may be administered intravenously, and the additional therapeutically active component may be administered orally). In any event, administering the components in a single dosage from, in separate dosage forms by the same route, or in separate dosage forms by different routes are all considered “concurrent administration,” for purposes of the present disclosure. For purposes of the present disclosure, administration of an anti-ACVR1 antibody “prior to”, “concurrent with,” or “after” (as those terms are defined herein above) administration of an additional therapeutically active component is considered administration of an anti-ACVR1 antibody “in combination with” an additional therapeutically active component.

The present invention includes pharmaceutical compositions in which an anti-ACVR1 antibody of the present invention is co-formulated with one or more of the additional therapeutically active component(s) as described elsewhere herein.

### Diagnostic Uses of the Antibodies

The antibodies of the present invention may be used to detect and/or measure ACVR1 protein in a sample, e.g., for diagnostic purposes. Some embodiments contemplate the use of one or more antibodies of the present invention in assays to detect a ACVR1-associated- or ACVR mutant-protein-associated-disease or disorder. Exemplary diagnostic assays for ACVR1 may comprise, e.g., contacting a sample, obtained from a patient, with an anti-ACVR1 antibody of the invention, wherein the anti-ACVR1 antibody is labeled with a detectable label or reporter molecule or used as a capture ligand to selectively isolate ACVR1 from patient samples. Alternatively, an unlabeled anti-ACVR1 antibody can be used in diagnostic applications in combination with a secondary antibody which is itself detectably labeled. The detectable label or reporter molecule can be a radioisotope, such as ³H, ¹⁴C, ³²P, ³⁵S, or ¹²⁵I; a fluorescent or chemiluminescent moiety such as fluorescein isothiocyanate, or rhodamine; or an enzyme such as alkaline phosphatase, β-galactosidase, horseradish peroxidase, or luciferase. Specific exemplary assays that can be used to detect or measure ACVR1 in a sample include enzyme-linked immunosorbent assay (ELISA), radioimmunoassay (RIA), and fluorescence-activated cell sorting (FACS).

Samples that can be used in ACVR1 diagnostic assays according to the present invention include any tissue or fluid sample obtainable from a patient, which contains detectable quantities of either ACVR1 protein, or fragments thereof, under normal or pathological conditions. Generally, levels of ACVR1 protein in a particular sample obtained from a healthy patient (e.g., a patient not afflicted with a disease associated with ACVR1) will be measured to initially establish a baseline, or standard, level of ACVR1. This baseline level of ACVR1 can then be compared against the levels of ACVR1 measured in samples obtained from individuals suspected of having a ACVR1-associated condition, or symptoms associated with such condition.

The antibodies specific for ACVR1 protein may contain no additional labels or moieties, or they may contain an N-terminal or C-terminal label or moiety. In one embodiment, the label or moiety is biotin. In a binding assay, the location of a label (if any) may determine the orientation of the peptide relative to the surface upon which the peptide is bound. For example, if a surface is coated with avidin, a peptide containing an N-terminal biotin will be oriented such that the C-terminal portion of the peptide will be distal to the surface.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1. Generation of Human Antibodies to Activin A Receptor 1 (ACVR1)

Human antibodies against ACVR1 (human and mouse cross-reactive) were isolated from a full length human IgG synthetic naive library using an in vitro yeast selection system and associated methods (Rouha H, et al. MAbs. 2015; 7(1):243-254). An antibody library of approximately 1×10¹⁰ in diversity was designed and propagated as described previously (Rouha et al. 2015, Xu et al., Protein Eng Des Sel. 2013; 26(10):663-670). ACVR1-binding antibodies were enriched by incubation of biotinylated ACVR1-Fc and Myc-His monomeric ACVR1 at different concentrations with antibody-expressing yeast cells followed by magnetic bead selection (Miltenyi Biotec) or flow cytometry on a FACSAria II cell sorter (BD Biosciences) using fluorescent streptavidin or extravidin secondary reagents in several successive selection rounds. Antibodies cross-reactive to off-target proteins ALK1, ALK3, and ALK6 were actively depleted from selection outputs. After the last round of enrichment, yeast cells were plated onto agar plates, analyzed by DNA sequencing, and expanded for IgG production. Heavy chains from the naive outputs were used to prepare light-chain diversification libraries, which were then used for additional selection rounds. In particular, heavy chains were extracted from the fourth naive selection round outputs and transformed into a light-chain library consisting of 1×10⁶ unique light chains to create new libraries approximately 1×10⁸ in total diversity. Antibody optimization was completed in 3 phases. Optimization of the heavy chain via diversification of the complementarity-determining regions (CDRs) HCDR1 and HCDR2 followed either by mutagenic PCR-based diversification of the entire heavy chain variable region or diversification of the light chain LCDR1 and LCDR2 segments. HCDR1and HCDR2 regions were diversified with premade libraries of HCDR1 and HCDR2 variants of a diversity of approximately 1×10⁸. Mutagenic PCR-based and premade libraries with LCDR1 and LCDR2 variants had diversities of approximately 1×10⁷ and 1×10⁵, respectively. Lead variants were further diversified via DNA oligonucleotide sequence variegation of the HCDR3 or LCDR3. Oligonucleotide HCDR3 and LCDR3 libraries had a diversity of approximately 1×10⁴ and 1×10³, respectively. Diversified antibody lineage populations were selected for enhanced binding to the target proteins while avoiding undesired cross-reactivity. The methods used for selections on diversified populations are similar or identical to those used to isolate the original lead IgGs (Xu et al., 2013).

Exemplary antibodies generated as disclosed above were designated as REGN 5166, REGN 5167, and REGN 5168. ACVR1 Fabs were generated from corresponding ACVR1 Mabs.

The biological properties of the exemplary antibodies generated in accordance with the methods of this Example are described in detail in the Examples set forth below.

### Example 2. Heavy and Light Chain Variable Region Amino Acid and Nucleotide Sequences

Table 1 sets forth the amino acid sequence identifiers of the heavy and light chain variable regions and CDRs of selected anti-ACVR1 antibodies of the invention.

TABLE 1
Amino Acid Sequence Identifiers

| Antibody | SEQ ID NO: | | | | | | | |
|---|---|---|---|---|---|---|---|---|
| Designation | HCVR | HCDR1 | HCDR2 | HCDR3 | LCVR | LCDR1 | LCDR2 | LCDR3 |
| REGN 5166 | 14 | 16 | 18 | 20 | 22 | 24 | 26 | 28 |
| REGN 5167 | 30 | 32 | 34 | 36 | 38 | 40 | 42 | 44 |
| REGN 5168 | 46 | 48 | 50 | 52 | 54 | 56 | 58 | 60 |

In Table 1, the amino acid sequence of LCDR2 SEQ ID NO: 26 is GAS; LCDR2 SEQ ID NO: 42 is GAS; and LCDR2 SEQ ID NO: 58 is KAS. The corresponding nucleic acid (DNA) sequence identifiers are set forth in Table 2.

TABLE 2
Nucleic Acid Sequence Identifiers

| Antibody | SEQ ID NO: | | | | | | | |
|---|---|---|---|---|---|---|---|---|
| Designation | HCVR | HCDR1 | HCDR2 | HCDR3 | LCVR | LCDR1 | LCDR2 | LCDR3 |
| REGN 5166 | 13 | 15 | 17 | 19 | 21 | 23 | 25 | 27 |
| REGN 5167 | 29 | 31 | 33 | 35 | 37 | 39 | 41 | 43 |
| REGN 5168 | 45 | 47 | 49 | 51 | 53 | 55 | 57 | 59 |

In Table 2, the DNA sequence of LCDR2 SEQ ID NO: 25 is GGCGCATCC; LCDR2 SEQ ID NO: 41 is GGTGCATCC; and LCDR2 SEQ ID NO: 57 is AAAGCCTCC.

Antibodies referred to herein typically have fully human variable regions, but may have human or mouse constant regions. As will be appreciated by a person of ordinary skill in the art, an antibody having a particular Fc isotype can be converted to an antibody with a different Fc isotype (e.g., an antibody with a mouse IgG1 Fc can be converted to an antibody with a human IgG4, etc.), but in any event, the variable domains (including the CDRs)—which are indicated by the numerical identifiers shown in Tables 1 or 2—will remain the same, and the binding properties to antigen are expected to be identical or substantially similar regardless of the nature of the Fc domain. In certain embodiments, selected antibodies with a mouse IgG1 Fc are converted to antibodies with human IgG4 Fc. In one embodiment, the IgG4 Fc domain comprises 2 or more amino acid changes as disclosed in US20100331527. In one embodiment, the human IgG4 Fc comprises a serine to proline mutation in the hinge region (S108P) to promote dimer stabilization. Unless indicated otherwise, all antibodies used in the following examples comprise a human IgG4 isotype.

Table 3 sets forth the nucleic acid (DNA) and amino acid (PEP) sequence identifiers of the heavy and light chains (HC and LC) of selected anti-ACVR1 antibodies of the invention.

TABLE 3
Sequence Identifiers for Heavy and Light Chains

| | SEQ ID NO: | | | |
|---|---|---|---|---|
| Antibody | HC | HC | LC | LC |
| Designation | DNA | PEP | DNA | PEP |
| REGN 5166 | 1 | 2 | 3 | 4 |
| REGN 5167 | 5 | 6 | 7 | 8 |
| REGN 5168 | 9 | 10 | 11 | 12 |

### Example 3. Antibody Binding to ACVR1 as Determined by Surface Plasmon Resonance

Biacore binding kinetics of anti-ACVR1 monoclonal antibodies binding to ACVR1 reagents was measured at 37° C. Reagent/mAb clones tested are shown in Table 4.

TABLE 4
Reagent/mAb Clone IDs:

| REGN# | Lot # | Description |
|---|---|---|
| REGN 5166 | REGN 5166-L1 | Bivalent hIgG4P |
| REGN 5167 | REGN 5167-L1 | Bivalent hIgG4P |
| REGN 5168 | REGN 5168-L1 | Bivalent hIgG4P |
| REGN 3111 | REGN 3111-L1 | hACVR1-.mmh |
| REGN 3407 | REGN 3407-L4 | mACVR1-mmh |
| REGN 3112 | REGN 3112-L1 | hACVR1-mFc |
| R&D System | 6506-RA-100 | mACVR1-mFc |

### Experimental Procedure

Equilibrium dissociation constants (K_{D}) for ACVR1 binding to purified anti-ACVR1 monoclonal antibodies were determined using a real-time surface plasmon resonance (SPR) based Biacore T200 biosensor. All binding studies were performed in 10 mM HEPES, 150 mM NaCl, 3 mM EDTA, and 0.05% v/v surfactant Tween-20, pH 7.4 (HBS-EP) running buffer at 37° C. The Biacore CM5 sensor surface was first derivatized by amine coupling with a monoclonal mouse anti-human Fc antibody (REGN2567) to capture anti-ACVR1 monoclonal antibodies. Different concentrations of ACVR1 reagents, human ACVR1 extracellular domain expressed with a C-terminal myc-myc-hexahistidine tag (hACVR1-mmh; REGN3111, SEQ ID NO: 63), mouse ACVR1 extracellular domain expressed with a C-terminal myc-myc-hexahistidine tag (mACVR1-mmh; REGN3407, SEQ ID NO: 65), human ACVR1 extracellular domain expressed with a C-terminal mouse IgG2a Fc tag (hACVR1-mFc; REGN3112, SEQ ID NO: 64); and mouse ACVR1 extracellular domain expressed with a C-terminal mouse IgG2a Fc tag (mACVR1-mFc, R&D system), at concentrations ranging from 0.41 nM to 100 nM in a series of 3-fold dilutions prepared in HBS-EP running buffer were injected at a flow rate of 50 μL/min for 1.5 minutes. The dissociation of different ACVR1 reagents bound to anti-ACVR1 monoclonal antibodies was monitored for 20 minutes in HBS-EP running buffer. At the end of each cycle, the anti-ACVR1 monoclonal antibodies capture surface was regenerated using a 12 sec injection of 20 mM H3PO4. The association rate (ka) and dissociation rate (kd) were determined by fitting the real-time binding sensorgrams to a 1:1 binding model with mass transport limitation using Scrubber 2.0c curve-fitting software. Binding dissociation equilibrium constant (KD) and dissociative half-life (t½) were calculated from the kinetic rates as:

$${K}_{D} ( M ) = \frac{kd}{ka} , and {t}^{1 / 2} ( min ) = \frac{ln ( 2 )}{60 * kd}$$

Binding kinetics parameters for different ACVR1 reagents to anti-ACVR1 monoclonal antibodies of the invention at 37° C. are shown in Table 5 through Table 8.

TABLE 5
Kinetic binding parameters for the interaction of hACVR1-
mmh with anti-ACVR1 monoclonal antibodies at 37° C.

| | mAb | 100 nM | | | | |
|---|---|---|---|---|---|---|
| | Capture | Ag | | | | |
| | Level | Bound | kₐ | k_{d} | K_{D} | t½ |
| REGN# | (RU) | (RU) | (1/Ms) | (1/s) | (M) | (min) |
| REGN 5166-L1 | 227 ± 0.2 | 43 | 6.77E+05 | 1.76E−04 | 2.60E−10 | 65 |
| REGN 5167-L1 | 238 ± 0.2 | 43 | 7.18E+05 | 1.63E−04 | 2.27E−10 | 71 |
| REGN 5168-L1 | 231 ± 0.5 | 42 | 1.31E+06 | 1.59E−03 | 1.21E−09 | 7 |

TABLE 6
Kinetic binding parameters for the interaction of hACVR1-
mFc with anti-ACVR1 monoclonal antibodies at 37° C.

| | mAb | 100 nM | | | | |
|---|---|---|---|---|---|---|
| | Capture | Ag | | | | |
| | Level | Bound | kₐ | k_{d} | K_{D} | t½ |
| REGN# | (RU) | (RU) | (1/Ms) | (1/s) | (M) | (min) |
| REGN 5166-L1 | 226 ± 0.2 | 142 | 1.18E+06 | 5.65E−05 | 4.78E−11 | 204 |
| REGN 5167-L1 | 236 ± 0.2 | 141 | 1.11E+06 | 5.36E−05 | 4.81E−11 | 215 |
| REGN 5168-L1 | 231 ± 0.4 | 145 | 1.87E+06 | 3.57E−04 | 1.91E−10 | 32 |

TABLE 7
Kinetic binding parameters for the interaction of mACVR1-
mmh with anti-ACVR1 monoclonal antibodies at 37° C.

| | mAb | 100 nM | | | | |
|---|---|---|---|---|---|---|
| | Capture | Ag | | | | |
| | Level | Bound | kₐ | k_{d} | K_{D} | t½ |
| REGN# | (RU) | (RU) | (1/Ms) | (1/s) | (M) | (min) |
| REGN 5166-L1 | 227 ± 0.6 | 43 | 6.74E+05 | 1.81E−04 | 2.68E−10 | 64 |
| REGN 5167-L1 | 237 ± 0.3 | 43 | 7.13E+05 | 1.61E−04 | 2.26E−10 | 72 |
| REGN 5168-L1 | 232 ± 0.2 | 42 | 1.34E+06 | 1.67E−03 | 1.24E−09 | 7 |

TABLE 8
Kinetic binding parameters for the interaction of mACVR1-
mFc with anti-ACVR1 monoclonal antibodies at 37° C.

| | mAb | 100 nM | | | | |
|---|---|---|---|---|---|---|
| | Capture | Ag | | | | |
| | Level | Bound | kₐ | k_{d} | K_{D} | t½ |
| REGN# | (RU) | (RU) | (1/Ms) | (1/s) | (M) | (min) |
| REGN 5166-L1 | 226 ± 0.3 | 134 | 2.03E+06 | 3.63E−05 | 1.79E−11 | 318 |
| REGN 5167-L1 | 236 ± 0.2 | 135 | 1.89E+06 | 3.63E−05 | 1.92E−11 | 318 |
| REGN 5168-L1 | 232 ± 0.4 | 135 | 3.06E+06 | 1.11E−04 | 3.62E−11 | 104 |

At 37° C., anti-ACVR1 monoclonal antibodies bound to hACVR1-mmh with K_{D} values ranging from 227 pM to 1.21 nM, as shown in Table 5.

At 37° C., anti-ACVR1 monoclonal antibodies bound to hACVR1-mFc with K_{D} values ranging from 47.8 pM to 191 pM, as shown in Table 6.

At 37° C., anti-ACVR1 monoclonal antibodies bound to mACVR1-mmh with K_{D} values ranging from 226 pM to 1.24 nM, as shown in Table 7.

At 37° C., anti-ACVR1 monoclonal antibodies bound to mACVR1-mFc with K_{D} values ranging from 17.9 pM to 36.2 pM, as shown in Table 8.

In order to measure the binding constants of ACVR1 Fabs, hACVR1.mmh was captured with a myc antibody (REGN642) immobilized on a CM5 chip. Different concentrations of ACVR1 Fabs were injected over hACVR1.mmh at 37° C. Binding rate constants and equilibrium dissociation rate constants were calculated by fitting data using 1:1 Langmuir binding model (Scrubber 2.0c). Binding constants of ACVR1 Fabs to human ACVR1 are shown in Table 9 (data for REGN 5168 Fab not shown).

TABLE 9
Binding constants of ACVR1 Fabs to hACVR1.mmh at 37° C.

| ACVR1 | ka | kd | KD | t1/2 |
|---|---|---|---|---|
| Fab tested | (1/Ms) | (1/s) | (M) | (min) |
| REGN 5167 Fab | 5.49E+05 | 9.36E−05 | 1.70E−10 | 123 |
| REGN 5166 Fab | 6.90E+05 | 1.06E−04 | 1.54E−10 | 109 |

All 3 ACVR1 Fabs bound to monomeric human ACVR1 with binding kinetics similar (<2.5-fold difference) to their respective mAbs.

In order to measure the binding constants of ACVR1 Fabs, mACVR1.mmh was captured with a myc antibody (REGN 642) immobilized on a CM5 chip. Different concentrations of ACVR1 Fabs were injected over mACVR1.mmh at 37° C. Binding rate constants and equilibrium dissociation rate constants were calculated by fitting data using 1:1 Langmuir binding model (Scrubber 2.0c). Binding constants of ACVR1 Fabs to mouse ACVR1 are shown in Table 10 (data for REGN 5168 Fab not shown).

TABLE 10
Binding constants of ACVR1 Fabs to mACVR1.mmh at 37° C.

| ACVR1 | kₐ | k_{d} | K_{D} | t_{1/2} |
|---|---|---|---|---|
| Fab tested | (1/Ms) | (1/s) | (M) | (min) |
| REGN 5167 Fab | 5.45E+05 | 9.72E−05 | 1.78E−10 | 119 |
| REGN 5166 Fab | 6.53E+05 | 1.05E−04 | 1.60E−10 | 110 |

All 3 ACVR1 Fabs bound to monomeric mouse ACVR1 with binding kinetics similar (<2.5-fold difference) to their respective mAbs.

### Example 4. Antibody Binding to ACVR1 Family Members as Determined by Surface Plasmon Resonance

ACVR1 mAbs were tested for cross-reactivity to a panel of bone morphogenic protein type 1 receptors including human and mouse ACVR1, BMPR1A, and BMPR1B, as well as ACVRL1 on a Biacore T200 as follows. In order to measure the specificity of ACVR1 mAbs, ACVR1 mAbs were captured with anti-human Fab immobilized on a CM5 chip. 100 nM or 10 nM of dimeric human or mouse ACVR1, ACVRL1, BMPR1A or BMPR1B were injected at 30 μL/min for 2 min (duplicate injections) and the experiment was performed at 25° C. Binding of different receptors to the captured mAb was determined using Scrubber 2.0c software. Results are shown in Table 11.

TABLE 11
ACVR1 antibodies are specific to ACVR1

| | mAb | | | | | | | | |
|---|---|---|---|---|---|---|---|---|---|
| | Capture | | | | | | | | |
| mAb | Level | 100 nM Human Receptor Binding | | | | 100 nM Mouse Receptor Binding | | | |
| Captured | (RU) | ACVR1 | ACVRL1 | BMPR1A | BMPR1B | ACVR1 | ACVRL1 | BMPR1A | BMPR1B |
| 5168 | 264 ± 4 | 147 ± 4 | 2 ± 0 | 2 ± 0.1 | 20 ± 0.6 | 140 ± 2.3 | 3 ± 0.4 | 8 ± 0.2 | 1 ± 0.2 |
| 5167 | 261 ± 0.5 | 130 ± 0.4 | 1 ± 0.1 | 2 ± 0.1 | 10 ± 0.5 | 130 ± 1.5 | 1 ± 0.1 | 3 ± 0.1 | 1 ± 0.1 |
| 5166 | 272 ± 2.2 | 147 ± 2.2 | 4 ± 0.1 | 2 ± 0 | 24 ± 0.6 | 141 ± 5.4 | 4 ± 0.4 | 9 ± 0.1 | 1 ± 0.1 |
| Isotype | 236 ± 0.8 | 11 ± 1.2 | 2 ± 0 | 1 ± 0.1 | 19 ± 0.5 | 8 ± 1 | 2 ± 0.2 | 7 ± 0.1 | 1 ± 0 |
| control | | | | | | | | | |

Results: None of the anti-ACVR1 mAbs bound to any of the other human or mouse receptors ACVRL1, BMPR1A, or BMPR1B, indicating that these mAbs REGN5166, REGN 5167, and REGN 5168 are specific to ACVR1, as shown in Table 11. The anti-ACVR1 mAbs REGN 5166, REGN 5167, and REGN 5168 bound to human and mouse ACVR1, as shown in Table 11.

### Example 5. Cross-Competition Analysis of anti-ACVR1 Antibodies

A cross-competition assay was conducted to assess the ability of a panel of 3 antibodies (REGN 5166, REGN 5167, and REGN 5168) to compete with one another for binding to human ACVR1. An isotype-matched that doesn't bind ACVR1 was used as a negative control.

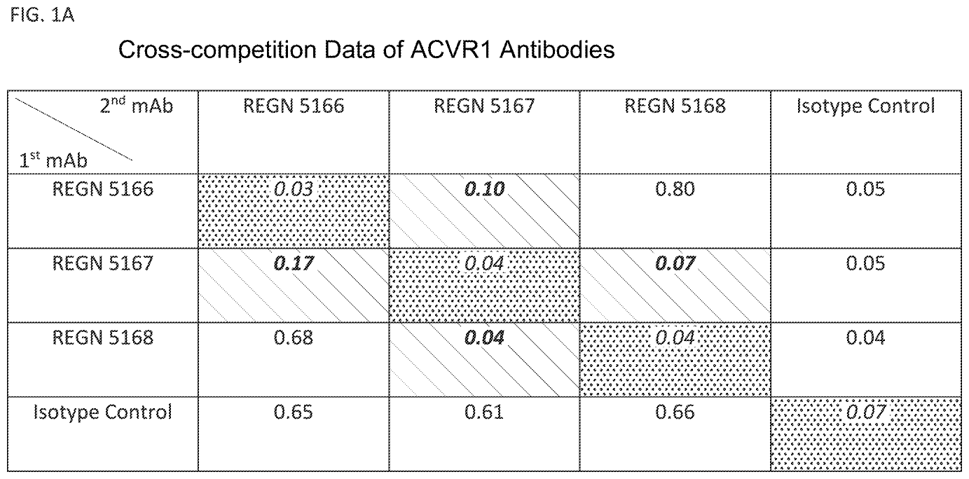

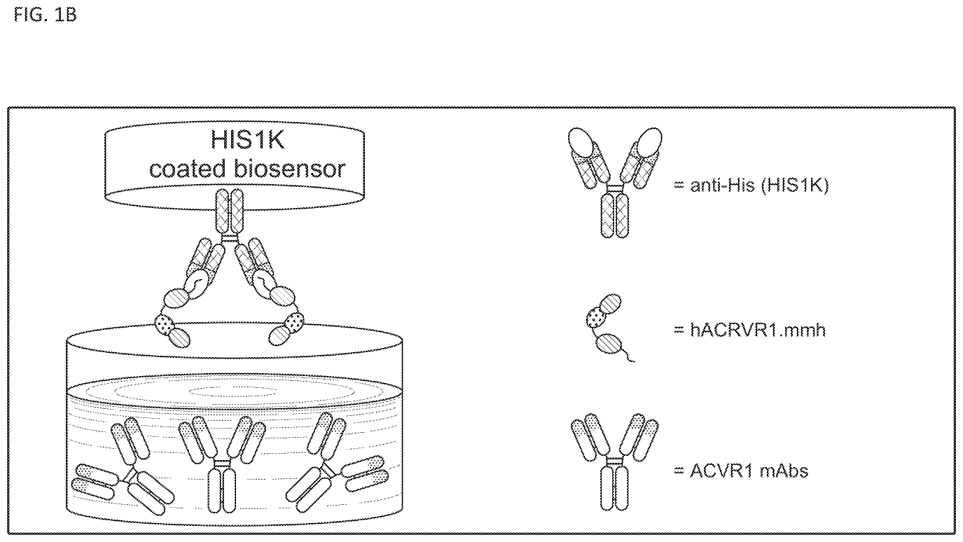

hACVR1.mmh was captured on a His antibody Octet® biosensor and was later saturated by dipping into wells containing 50 μg/mL of ACVR1 mAbs (referred to as 1st mAb) for 4 min, as illustrated in FIG. 1B. Then, 1st mAb saturated biosensors were dipped into the wells containing 50 μg/mL of different ACVR1 mAbs (referred to as 2nd mAb) for 3 min. The binding of 2nd mAb to the complex of hACVR1.mmh and 1st mAb was determined, and the observed wavelength shift (nm) is reported in FIG. 1A. The shaded cells represent bidirectional competition while white cells represent no competition.

These data indicate that REGN 5166 and REGN 5167 recognize partially overlapping epitopes (or, alternatively, that binding of one of them to ACVR1 sterically hinders the binding of the other); and that REGN 5168 and REGN 5167 likely bind overlapping epitopes. Note that there is no cross-competition between REGN 5168 and REGN 5166, indicating that the epitope recognized by REGN 5168 is distinct from that recognized by REGN 5166.

### Example 6. Cell Binding by Flow Cytometry with HEK293/hACVR1-R206H Cells

In order to assess cell binding by anti-hACVR1 antibodies of the invention, HEK293 cells (human embryonic kidney, ATCC) were engineered to stably overexpress full length human ACVR1 (amino acids 1-509, R206H, of accession #Q04771) as well as a BMP-response element fused to firefly luciferase reporter (BRE-Luc). A single clone of the cell line was isolated, and the resulting cell line was named HEK293/BRE-luc/hACVR1-R206H-clone H2. It is hereafter referred to as HEK293/hACVR1-R206H.

To assess binding of the anti-ACVR1 antibodies of the invention to ACVR1 receptors expressed on the cell surface, 70 nM of the antibodies were incubated with 5×10⁵ cells/well at 4° C. for 30 minutes in PBS (without calcium and magnesium) containing 2% FBS (flow buffer). After incubation with primary antibodies, the cells were stained with 3.2 μg/mL of Alexa Fluor®-647 conjugated secondary antibody (Jackson ImmunoResearch Laboratories Inc., anti-human #109-607-003) at 4° C. for 25 minutes. Cells were fixed using BD CytoFix™ (Becton Dickinson, # 554655) and analyzed on IQue® Flow Cytometer (Intellicyt®). Unstained and secondary antibody alone controls were also tested on cells. The results were analyzed using ForeCyt® (IntelliCyt®) software to determine the geometric means of fluorescence for viable cells and the binding ratio was calculated by normalizing the geometric mean value of the test condition by the geometric mean value of the corresponding unstained cells. Results are shown in Table 12.

TABLE 12
Binding of anti-hACVR1 antibodies to HEK293/hACVR1-R206H cells

| | MFI—Normalized to Unstained | |
|---|---|---|
| | Control | |
| | HEK293 | HEK293/hAC |
| REGN# | Parental | VR1-R206H |
| REGN 5166 | 34 | 2005 |
| REGN 5167 | 27 | 1868 |
| REGN 5168 | 28 | 2247 |
| hIgG Control | 1 | 3 |
| Anti-Human 2″ alone | 1 | 1 |

As shown in Table 12, all three of the antibodies of the invention bound to HEK293/hACVR1-R206H cells with binding ratios ranging from 1868 to 2247-fold. Antibodies of the invention bound to HEK293 parental cells with binding ratios 27 to 34-fold. The human IgG control antibody and secondary antibody alone samples demonstrated binding ratios ranging from 1 to 3-fold.

### Example 7. Functional Inhibition of hACVR1 Signaling in a Cell-Based Bioassay Using HEK293/BRE-luc/hACVR1-R206H-clone H2 Cells Activated by hBMP7

Activin A receptor type I, ACVR1 (also known as ActRI, ACVR1A, or Alk2), is a single-pass transmembrane receptor and a member of the type I BMP receptor of the TGF-13 receptor super family. Upon ligand binding, ACVR1 interacts with type II receptors causing signal transduction (Massague 1998, Massague et al. 2005).

In order to assess anti-ACVR1 antibody-mediated inhibition of ACVR1, a bioassay was established using HEK293 cells (human embryonic kidney, ATCC) stably overexpressing full length human ACVR1 (amino acids 1-509, R206H, of accession #Q04771) as well as a BMP-response element fused to firefly luciferase reporter (BRE-Luc). HEK293 cells endogenously express receptors and other components of the BMP signal transduction machinery. A single clone of the cell line was isolated and the resulting cell line was named HEK293/BRE-luc/hACVR1-R206H-clone H2. It is hereafter referred to as HEK293/BRE-luc/hACVR1-R206H.

For the bioassay, HEK293/BRE-luc/hACVR1-R206H cells were plated at 10,000 cells/well in a 96-well plate in assay buffer (DMEM High Glucose+10% FBS+Pen/Strep/L-Glutamine) and incubated at 37° C. in 5% CO₂. After 5 hours, anti-hACVR1 antibodies or a human IgG control antibody were serially diluted 1:4 in assay buffer from 300 nM to 73.2 pM (plus a sample containing buffer alone without test molecule) and then added to the cells and incubated at 25° C. for 30 minutes. After 30 minutes, 2 nM human Bone Morphogenetic Protein 7 (hBMP7, R&D System 354-BP/C) was added to the cells. To obtain a dose dependent activation by the ligand, hBMP7 was serially diluted 1:3 from 100 nM to 1.7 pM in assay buffer (plus a sample containing buffer alone without test molecule) and added to cells alone. After overnight incubation at 37° C. in 5% CO₂, luciferase activity was measured with OneGlo™ reagent (Promega, # E6031) and Envision plate readers (Perkin Elmer). The results were analyzed using nonlinear regression (4-parameter logistics) with Prism software (GraphPad) to obtain EC₅₀ and IC₅₀ values. The percentage of inhibition was calculated with the RLU values using the following equation:

$$Maximum Percent inhibition = \frac{{RLU}_{Baseline} - {RLU}_{Experimental}}{{RLU}_{Baseline} - {RFU}_{Background}} \times 100$$

In this equation “RLU_{Baseline}” is the luminescence value from the cells treated with 2 nM hBMP7 without antibodies, “RLU_{Experimenal}” are the lowest luminescence values recorded across the tested range of antibody concentrations, and “RLU_{Background}” is the luminescence value from cells without any ligands or antibodies. Results are shown in Table 13.

TABLE 13
Inhibition of anti-hACVR1 antibodies in presence of 2 nM
hBMP7 in HEK293/BRE-luc/hACVR1-R206H cells

| Ligand | hBMP7 | |
|---|---|---|
| EC₅₀ [M] | 2.97E−10 | |
| Constant Ligand | 2 nM hBMP7 | |
| Concentration | | |
| | | Max. % |
| REGN# | IC₅₀ [M] | Inhibition |
| REGN 5166 | 1.1E−09 | 110 |
| REGN 5167 | 1.3E−09 | 111 |
| REGN 5168 | 9.8E−10 | 109 |
| hIgG Control | Not | 39.5 |
| | Determined | |

### Results Summary and Conclusions

Three anti-human ACVR1 antibodies of the invention were tested for their ability to inhibit BMP-7 mediated activation of HEK293/BRE-luc/hACVR1-R206H cells. As shown in Table 13, each of the antibodies of the invention showed 109-111% inhibition of 2 nM hBMP7 with IC₅₀ values ranging from 980 pM to 1.3 nM. An IgG control antibody showed 39.5% inhibition of 2 nM hBMP7 with an IC₅₀ value that could not be determined because concentration dependent activity was not observed within the tested range of concentrations. A dose response of hBMP7 determined that BMP7 activated HEK293/BRE-luc/hACVR1-R206H cells with an EC₅₀ value of 1.22 nM.

### References

Massagué J, TGF-beta Signal Transduction, Annu. Rev. Biochem. 1998. 67:753-91, PMID: 9759503

Massagué J, Seoane J, Wotton D, Smad transcription factors, Genes Dev. 2005 19: 2783-2810, PMID: 16322555

### Example 8. Trauma-Induced Heterotopic Ossification Studies in Burn and Achilles Tenotomy Mouse Model

A mouse model of trauma-induced heterotopic ossification was employed to evaluate anti-ACVR1 antibodies of the disclosure. C57BI/6 (WT) and transgenic No MAHA mice (8-10 weeks old) were obtained from commercial vendors or Velocigene®. “No MAHA” are specialized mice engineered to minimize an anti-human antibody immune response. Mice were acclimated for 2 days in the Regeneron Animal Facility before the initiation of experiments. Mice were anesthetized according to protocol using inhaled isoflurane. Buprenorphine ER was administered once prior to surgery (sustained release over 72 hours), and second dose at 72 hrs. Dorsal hair was trimmed using an automatic clipper and the surgical site was prepared aseptically with 3 alternating applications of povidone iodine and alcohol. A custom-made rectangular block (2×3 cm) was preheated in a 60° C. water bath. The block was applied to the dorsum of the mouse and held in place for 30 s to create a 30% total body surface area, partial thickness scald burn. The block was placed at the midpoint of the distance between the base of the cervical spine and lumbosacral spine. Sham animals underwent the same procedure, but the mold was placed in a 30° C. water bath instead. Post burn injury, each mouse was resuscitated with 1 ml of warm saline solution through an intraperitoneal (IP) injection and 0.5 ml of normal saline in a subcutaneous (SC) injection.

All mice (except sham animals) received an Achilles tenotomy on one hind limb, which in simple terms is a surgical cut of the Achilles tendon. Skin at the site of Achilles tenotomy is prepared for aseptic surgery (a 10% povidone-iodine scrub followed by a 70% alcohol wipe repeated three times). A sterile iris scissors was used to make a 1 cm skin incision along the lateral aspect of the

Achilles tendon. The Achilles tendon was aseptically exposed from the distal portion of the gastrocnemius muscle to the insertion on the calcaneus. The tendon was divided sharply at its midpoint and the skin incision was closed with non-absorbable suture such as nylon and then sutures removed in 10-14 days or tissue adhesive. Mice were weighed and wounds inspected daily for signs of infection.

The Achilles Tenotomy and Burn injury were done on the same day, one procedure following the other, where the order does not matter.

MicroCT (micro-computed tomography) scans were performed on the Quantum GX microCT Imaging System (Revvity Inc.) and scan parameters were: 18 sec scan, 90 um voxel size, 45 mm FOV.

For prophylactic/delayed dosing studies, total heterotopic bone volume was measured as follows:

Total HO=[volume of calcaneus(HO+native skeleton)−calcaneus_{contralateral}]+HO_{unattached}(HOₕₑₑₗ+HO_{calf})

### Prophylactic Dosing Study in C57BL/6 Mice

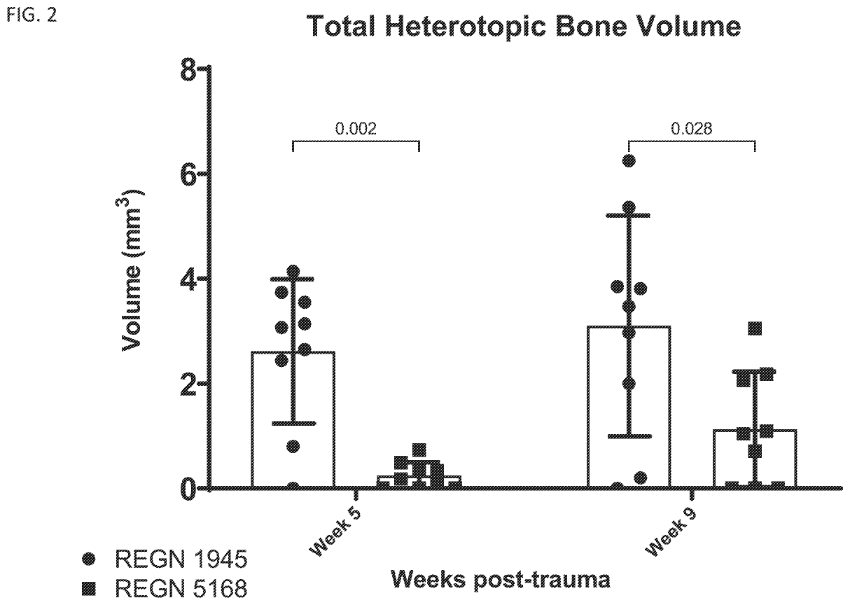

To confirm that ACVR1 antibodies can inhibit WT ACVR1 in the setting of HO in vivo, anti-ACVR1 antibody REGN 5168 was tested in the burn tenotomy model of trauma-induced HO (tHO) in WT mice. A prophylactic dosing study was performed in C57BLJ6 mice. Mice were dosed once weekly subcutaneously with 25 mg/kg of anti-ACVR1 antibody REGN 5168 (n=9) or REGN 1945 (n=9, isotype control) for 12 weeks post-trauma (Achilles tenotomy and burn injury) for a total of 13 doses. MicroCT (Quantum GX, Revvity) scans of the hind limbs were obtained at weeks 5 and 9 post-trauma and used to perform quantification of the heterotopic bone volume. Results are shown in Table 14 and FIG. 2.

TABLE 14
Total HO in Prophylactic Dosing Experiment in C57BL/6 Mice

| | Total HO (mm3) (Mean ± SD) | |
|---|---|---|
| | Week 5 | Week 9 |
| REGN 1945 | 2.614 ± 1.374 | 3.101 ± 2.106 |
| REGN 5168 | 0.242 ± 0.255 | 1.126 ± 1.099 |

Results: Inhibition of ACVR1 with REGN 5168 significantly reduced total heterotopic bone formation compared to isotype control in the Achilles tenotomy and burn injury in wild-type mice at 5 weeks and 9 weeks post-trauma in prophylactic treatment as shown in Table 14 and FIG. 2. These results confirm that antibody-mediated inhibition of WT ACVR1 blocks tHO.

Serum hepcidin and iron measurements. Serum hepcidin was measured using a murine hepcidin ELISA kit (Intrinsic Biosciences) following the manufacturer's protocol from serum obtained from terminal blood drawn at euthanasia. Serum iron was measured using the QuantiChrom Iron Assay Kit (BioAssay Systems, DIFE-250) following the manufacturer's protocol from serum obtained from terminal blood draw at euthanasia.

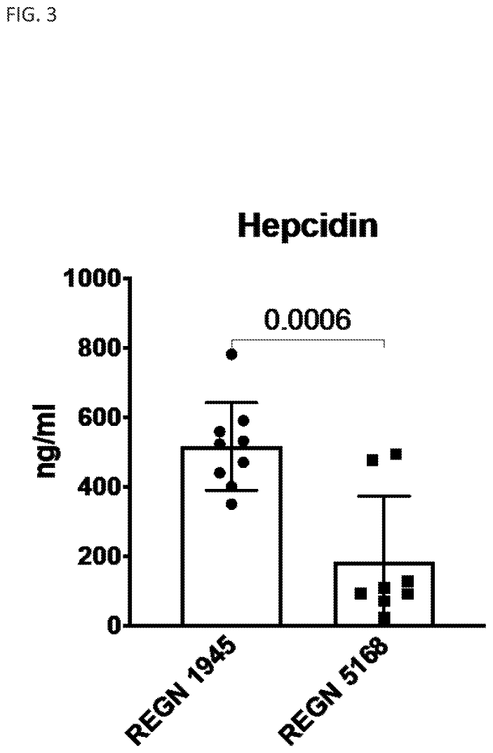

Results: Serum hepcidin was significantly decreased in WT mice following prophylactic treatment with REGN 5168, compared to isotype control treated mice as shown in FIG. 3. Mean serum iron was increased in WT mice following prophylactic treatment with REGN 5168, compared to isotype control treated WT mice, but data trend did not reach significance in this experiment (data not shown). These data demonstrate that the same ACVR1 antibody that inhibits wild type ACVR1 in vivo can extend its physiological effects to a system other than HO. ACVR1 antibodies may be considered as a therapeutic option for trauma-induced HO and could also be considered in conditions where increasing iron levels is desirable.

### Prophylactic Dosing Study in No MAHA Mice

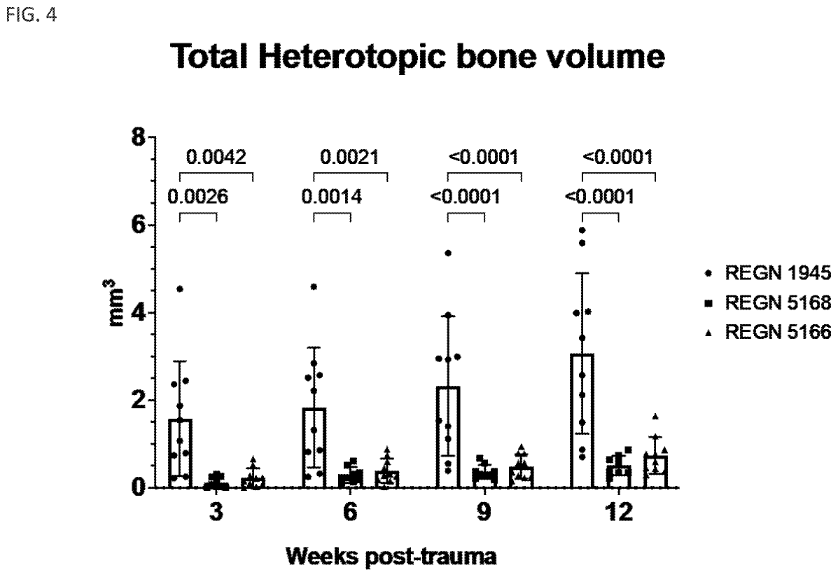

A prophylactic dosing study was performed in No MAHA transgenic mice. Mice were dosed once weekly subcutaneously with 25 mg/kg of REGN 1945 isotype control, or anti-ACVR1 antibodies REGN 5168 or REGN 5166. (n=9 each group). The REGN 5166 group received 8 antibody doses, of which the first 2 doses were 12.5 mg/kg. Mice treated with REGN 1945 or REGN 5168 received 12 doses total. MicroCT (Quantum GX, Revvity) scans of the hind limbs were obtained at Weeks 3, 6, 9 and 12 post-trauma, and used to perform quantification of the heterotopic bone volume. Results are shown in Table 15 and FIG. 4.

TABLE 15
Total HO in prophylactic Dosing Study Experiment in No MAHA Mice
Total HO (mm³) (Mean ± SD)

| | Week 3 | Week 6 | Week 9 | Week 12 |
|---|---|---|---|---|
| REGN 1945 | 1.582 ± 1.308 | 1.830 ± 1.366 | 2.316 ± 1.593 | 3.065 ± 1.833 |
| REGN 5168 | 0.123 ± 0.123 | 0.291 ± 0.177 | 0.363 ± 0.157 | 0.513 ± 0.225 |
| REGN 5166 | 0.225 ± 0.215 | 0.387 ± 0.275 | 0.487 ± 0.276 | 0.738 ± 0.416 |

Results: Inhibition of ACVR1 with REGN 5168 or REGN 5166 significantly reduced total heterotopic bone formation compared to isotype control in the Achilles tenotomy and burn injury in No MAHA transgenic mice at 3 weeks, 6 weeks, 9 weeks, and 12 weeks post-trauma in prophylactic treatment as shown in Table 15 and FIG. 4.

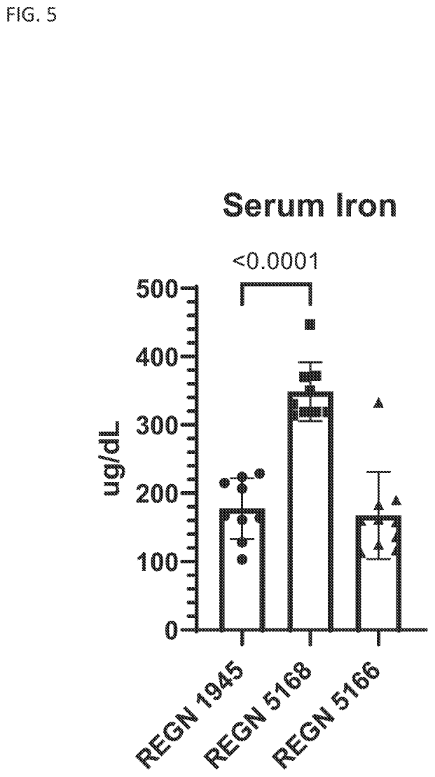

Serum iron in No MAHA mice was measured using the QuantiChrom™ Iron Assay Kit (BioAssay Systems, DIFE-250) following the manufacturer's protocol in serum derived from terminal blood draw at euthanasia. Results: Serum iron was significantly increased in No MAHA mice receiving REGN 5168, but not those receiving REGN 5166, compared to mice receiving isotype control REGN 1945, as shown in FIG. 5.

### Delayed Dosing Study in C57BL/6 Mice

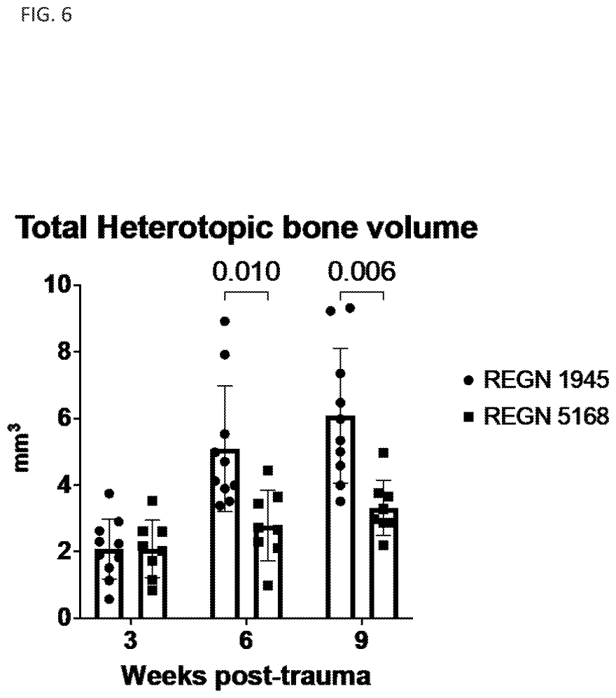

A delayed dosing study was performed in C57BL/6 mice. For the delayed dosing study, mice underwent the Achilles tenotomy and burn injury and were allowed to develop heterotopic bone until Week 3, at which point they were divided into either REGN 1945 (n=10) or REGN 5168 (n=8) treatment groups. Mice were treated once weekly at 25 mg/kg subcutaneously, for 8 weeks from the start of the treatment, at which point treatment was ceased. HO bone volume was measured using microCT scans of the hind limbs at Weeks 3, 6, 9. Results are shown in Table 16 and FIG. 6.

TABLE 16
Total HO in Delayed Dosing Study in C57BL/6 Mice

| | Total HO (mm³) (Mean ± SD) | | |
|---|---|---|---|
| | Week 3 | Week 6 | Week 9 |
| REGN 1945 | 2.073 ± 0.907 | 5.094 ± 1.887 | 6.077 ± 2.031 |
| REGN 5168 | 2.080 ± 0.862 | 2.780 ± 1.059 | 3.316 ± 0.828 |

Results: Inhibition of ACVR1 with REGN 5168 significantly reduced total heterotopic bone formation compared to isotype control in the Achilles tenotomy and burn injury in wild-type mice at 6 weeks and 9 weeks post-trauma in delayed treatment study as shown in Table 16 and FIG. 6.

### Example 9. Heterotopic Bone Resection Study Mouse Model

A surgical resection study was performed in No MAHA transgenic mice.

Mice in the resection study were allowed to develop heterotopic bone for 7 weeks post-trauma (Achilles tenotomy and burn injury), at which point the heterotopic bone was resected, and mice were divided into REGN 1945 or REGN 5168 treatment groups.

Mice were anesthetized using inhaled isoflurane or Ketamine and Xylazine, after the onset of anesthesia, Buprenorphine ER was administered, skin at the location of ectopic bone is shaved and the exposed skin prepared for aseptic surgery (a 10% povidone-iodine scrub followed by a 70% alcohol wipe repeated three times). A small incision (-1 cm) was made at the site of the ectopic bone lesion and the lesion exposed. An individual ectopic bone lesion was resected with as little surrounding muscle and soft tissue as possible. Hemostasis was achieved by hemostat pressure for 1-2 minutes or cauterization if needed. The incision was closed with (5-0 Vicryl) absorbable suture in an interrupted pattern or wound clips or tissue adhesive).

Mice received once weekly doses at 25 mg/kg of either REGN 1945 or REGN 5168 treatment for a total of 11 doses. Monitored HO progression by microCT scans of the hind limbs were obtained at Weeks 3, 6, at week 7-before and after-resection, 9, 12, 15 and 18 post-trauma.

Regrowth of Heterotopic bone was assessed by in vivo microCT imaging. MicroCT scans were performed on the Quantum GX (Perkin Elmer) and scan parameters were: 18 sec scan, 90 um voxel size, 45 mm FOV.

For resection studies, Total heterotopic bone volume was defined as follows:

Total HO=[volume of calcaneus(HO+native skeleton)]+HO_{unattached}(HOₕₑₑₗ+HO_{calf})

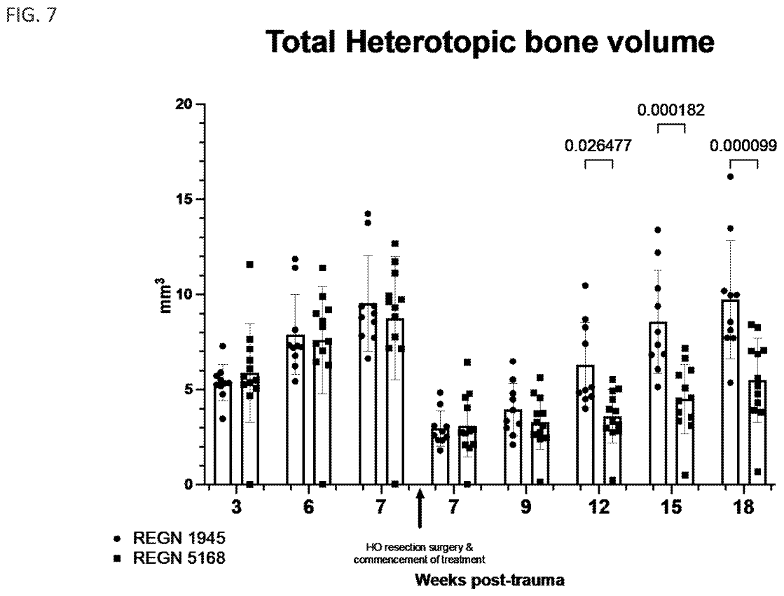

Results are shown in Table 17 and FIG. 7.

TABLE 17
Total HO in Surgical Resection Study Experiment in No MAHA Mice

| | Total HO (mm³) (Mean ± SD) | | | | | |
|---|---|---|---|---|---|---|
| | Week 3 | Week 6 | Week 9* | Week 12 | Week 15 | Week 18 |
| REGN 1945 | 5.379 ± 0.951 | 7.897 ± 2.103 | 3.951 ± 1.375 | 6.299 ± 2.233 | 8.565 ± 2.705 | 9.731 ± 3.114 |
| REGN 5168 | 5.884 ± 2.609 | 7.599 ± 2.802 | 3.282 ± 1.419 | 3.593 ± 1.407 | 4.502 ± 1.827 | 5.498 ± 2.202 |

*HO resection surgery and treatment started at week 7 post-trauma.

Results: Inhibition of ACVR1 with neutralizing antibody REGN 5168 in the Achilles tenotomy and burn injury tHO mouse model significantly inhibits HO recurrence at week 12, week 15, and week 18 post-trauma (week 5, week 8, and week 11 post-resection) in No MAHA mice as shown in Table 17 and FIG. 7.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Sequences
SEQ ID NO: 1
caggtgcagc tggtggagtc tgggggaggc gtagtccagc ctgggaggtc cctgagactc 60
tcctgtgcag cgtctggatt caccttcagt gcgtattgga tgcactgggt ccgccaggct 120
ccaggcaagg ggctggagtg ggtggcagtt atatcgtatg atggaagtaa taaatactat 180
gcagactccg tgaagggccg attcaccatc tccagagacc attccaagaa cacgctgtat 240
ctgcaaatga acagcctgag agccgaggac acggcggtgt actactgcgc aaaaggggac 300
gcctacgact cctggaggat actactcggc ggagattact acggcatgga tgtttggggc 360
cagggaacaa ctgtcaccgt ctcctcagcc tccaccaagg gcccatcggt cttccccctg 420
gcgccctgct ccaggagcac ctccgagagc acagccgccc tgggctgcct ggtcaaggac 480
tacttccccg aaccggtgac ggtgtcgtgg aactcaggcg ccctgaccag cggcgtgcac 540
accttcccgg ctgtcctaca gtcctcagga ctctactccc tcagcagcgt ggtgaccgtg 600
ccctccagca gcttgggcac gaagacctac acctgcaacg tagatcacaa gcccagcaac 660
accaaggtgg acaagagagt tgagtccaaa tatggtcccc catgcccacc ctgcccagca 720
cctgagttcc tggggggacc atcagtcttc ctgttccccc caaaacccaa ggacactctc 780
atgatctccc ggacccctga ggtcacgtgc gtggtggtgg acgtgagcca ggaagacccc 840
gaggtccagt tcaactggta cgtggatggc gtggaggtgc ataatgccaa gacaaagccg 900
cgggaggagc agttcaacag cacgtaccgt gtggtcagcg tcctcaccgt cctgcaccag 960
gactggctga acggcaagga gtacaagtgc aaggtctcca acaaaggcct cccgtcctcc 1020
atcgagaaaa ccatctccaa agccaaaggg cagccccgag agccacaggt gtacaccctg 1080
cccccatccc aggaggagat gaccaagaac caggtcagcc tgacctgcct ggtcaaaggc 1140
ttctacccca gcgacatcgc cgtggagtgg gagagcaatg ggcagccgga gaacaactac 1200
aagaccacgc ctcccgtgct ggactccgac ggctccttct tcctctacag caggctcacc 1260
gtggacaaga gcaggtggca ggaggggaat gtcttctcat gctccgtgat gcatgaggct 1320
ctgcacaacc actacacaca gaagtccctc tccctgtctc tgggtaaatg a 1371
SEQ ID NO: 2
QVQLVESGGG VVQPGRSLRL SCAASGFTFS AYWMHWVRQA PGKGLEWVAV ISYDGSNKYY 60
ADSVKGRFTI SRDHSKNTLY LQMNSLRAED TAVYYCAKGD AYDSWRILLG GDYYGMDVWG 120
QGTTVTVSSA STKGPSVFPL APCSRSTSES TAALGCLVKD YFPEPVTVSW NSGALTSGVH 180
TFPAVLQSSG LYSLSSVVTV PSSSLGTKTY TCNVDHKPSN TKVDKRVESK YGPPCPPCPA 240
PEFLGGPSVF LFPPKPKDTL MISRTPEVTC VVVDVSQEDP EVQFNWYVDG VEVHNAKTKP 300
REEQFNSTYR VVSVLTVLHQ DWLNGKEYKC KVSNKGLPSS IEKTISKAKG QPREPQVYTL 360
PPSQEEMTKN QVSLTCLVKG FYPSDIAVEW ESNGQPENNY KTTPPVLDSD GSFFLYSRLT 420
VDKSRWQEGN VFSCSVMHEA LHNHYTQKSL SLSLGK 456
SEQ ID NO: 3
gaaattgtgt tgacgcagtc tccaggcacc ctgtctttgt ctccagggga aagagccacc 60
ctctcctgca gggccagtca gagtgttagc agcagctact tagcctggta ccagcagaaa 120
cctggccagg ctcccaggcc cctcatctat ggcgcatcca gcagggccac tggcatccca 180
gacagattca gtggcagtgg gtctgggaca gacttcactc tcaccatcag cagactggag 240
cctgaagatt ttgcagtgta ttactgtcag cagggggggg acgcccctcc ttacactttc 300
ggcggaggga ccaaggttga gatcaaacga actgtggctg caccatctgt cttcatcttc 360
ccgccatctg atgagcagtt gaaatctgga actgcctctg ttgtgtgcct gctgaataac 420
ttctatccca gagaggccaa agtacagtgg aaggtggata acgccctcca atcgggtaac 480
tcccaggaga gtgtcacaga gcaggacagc aaggacagca cctacagcct cagcagcacc 540
ctgacgctga gcaaagcaga ctacgagaaa cacaaagtct acgcctgcga agtcacccat 600
cagggcctga gctcgcccgt cacaaagagc ttcaacaggg gagagtgtta g 651
SEQ ID NO: 4
EIVLTQSPGT LSLSPGERAT LSCRASQSVS SSYLAWYQQK PGQAPRPLIY GASSRATGIP 60
DRFSGSGSGT DFTLTISRLE PEDFAVYYCQ QGGDAPPYTF GGGTKVEIKR TVAAPSVFIF 120
PPSDEQLKSG TASVVCLLNN FYPREAKVQW KVDNALQSGN SQESVTEQDS KDSTYSLSST 180
LTLSKADYEK HKVYACEVTH QGLSSPVTKS FNRGEC 216
SEQ ID NO: 5
cagctgcagc tgcaggagtc gggcccagga ctggtgaagc cttcggagac cctgtccctc 60
acctgcactg tctctggtgg ctccatcacg agtagtagtt actactgggc gtggatccgc 120
cagcccccag ggaaggggct ggagtggatt gggaagatct attatagtgg gagcacccat 180
tacaacccgt ccctcaagag tcgagtcacc atatccgtag acacgtccaa gaaccagttc 240
tccctgaagt tgagttctgt gaccgccgca gacacggcgg tgtactactg cgccagagtt 300
ggaggacacg gctacggaga ctccgcaggg ttagccttcg atatctgggg tcagggtaca 360
atggtcaccg tctcctcagc ctccaccaag ggcccatcgg tcttccccct ggcgccctgc 420
tccaggagca cctccgagag cacagccgcc ctgggctgcc tggtcaagga ctacttcccc 480
gaaccggtga cggtgtcgtg gaactcaggc gccctgacca gcggcgtgca caccttcccg 540
gctgtcctac agtcctcagg actctactcc ctcagcagcg tggtgaccgt gccctccagc 600
agcttgggca cgaagaccta cacctgcaac gtagatcaca agcccagcaa caccaaggtg 660
gacaagagag ttgagtccaa atatggtccc ccatgcccac cctgcccagc acctgagttc 720
ctggggggac catcagtctt cctgttcccc ccaaaaccca aggacactct catgatctcc 780
cggacccctg aggtcacgtg cgtggtggtg gacgtgagcc aggaagaccc cgaggtccag 840
ttcaactggt acgtggatgg cgtggaggtg cataatgcca agacaaagcc gcgggaggag 900
cagttcaaca gcacgtaccg tgtggtcagc gtcctcaccg tcctgcacca ggactggctg 960
aacggcaagg agtacaagtg caaggtctcc aacaaaggcc tcccgtcctc catcgagaaa 1020
accatctcca aagccaaagg gcagccccga gagccacagg tgtacaccct gcccccatcc 1080
caggaggaga tgaccaagaa ccaggtcagc ctgacctgcc tggtcaaagg cttctacccc 1140
agcgacatcg ccgtggagtg ggagagcaat gggcagccgg agaacaacta caagaccacg 1200
cctcccgtgc tggactccga cggctccttc ttcctctaca gcaggctcac cgtggacaag 1260
agcaggtggc aggaggggaa tgtcttctca tgctccgtga tgcatgaggc tctgcacaac 1320
cactacacac agaagtccct ctccctgtct ctgggtaaat ga 1362
SEQ ID NO: 6
QLQLQESGPG LVKPSETLSL TCTVSGGSIT SSSYYWAWIR QPPGKGLEWI GKIYYSGSTH 60
YNPSLKSRVT ISVDTSKNQF SLKLSSVTAA DTAVYYCARV GGHGYGDSAG LAFDIWGQGT 120
MVTVSSASTK GPSVFPLAPC SRSTSESTAA LGCLVKDYFP EPVTVSWNSG ALTSGVHTFP 180
AVLQSSGLYS LSSVVTVPSS SLGTKTYTCN VDHKPSNTKV DKRVESKYGP PCPPCPAPEF 240
LGGPSVFLFP PKPKDTLMIS RTPEVTCVVV DVSQEDPEVQ FNWYVDGVEV HNAKTKPREE 300
QFNSTYRVVS VLTVLHQDWL NGKEYKCKVS NKGLPSSIEK TISKAKGQPR EPQVYTLPPS 360
QEEMTKNQVS LTCLVKGFYP SDIAVEWESN GQPENNYKTT PPVLDSDGSF FLYSRLTVDK 420
SRWQEGNVFS CSVMHEALHN HYTQKSLSLS LGK 453
SEQ ID NO: 7
gaaattgtgt tgacgcagtc tccaggcacc ctgtctttgt ctccagggga aagagccacc 60
ctctcctgca gggccagtca gagtgttagc agcgccttct tagcctggta ccagcagaaa 120
cctggccagg ctcccaggct cctcatctat ggtgcatcct acaggcacac tggcatccca 180
gacaggttca gtggcagtgg gtctgggaca gacttcacto tcaccatcag cagactggag 240
cctgaagatt ttgcagtgta ttactgtcag cactacggag ttggtcctag gactttcggc 300
ggagggacca aggttgagat caaacgaact gtggctgcac catctgtctt catcttcccg 360
ccatctgatg agcagttgaa atctggaact gcctctgttg tgtgcctgct gaataacttc 420
tatcccagag aggccaaagt acagtggaag gtggataacg ccctccaatc gggtaactcc 480
caggagagtg tcacagagca ggacagcaag gacagcacct acagcctcag cagcaccctg 540
acgctgagca aagcagacta cgagaaacac aaagtctacg cctgcgaagt cacccatcag 600
ggcctgagct cgcccgtcac aaagagcttc aacaggggag agtgttag 648
SEQ ID NO: 8
EIVLTQSPGT LSLSPGERAT LSCRASQSVS SAFLAWYQQK PGQAPRLLIY GASYRHTGIP 60
DRFSGSGSGT DFTLTISRLE PEDFAVYYCQ HYGVGPRTFG GGTKVEIKRT VAAPSVFIFP 120
PSDEQLKSGT ASVVCLLNNF YPREAKVQWK VDNALQSGNS QESVTEQDSK DSTYSLSSTL 180
TLSKADYEKH KVYACEVTHQ GLSSPVTKSF NRGEC 215
SEQ ID NO: 9
caggtgcagc tggtgcagtc tggggctgag gtgaaggagc ctggggcctc agtgaaggtt 60
tcctgcaagg catctggata caccttcgcg gagtactata tgcactgggt gcgacaggcc 120
cctggacaag ggcttgagtg gatgggagcg attaacccta gtggtagtca tacaagctac 180
gcacagaagt tccagggcag agtcaccatg accagggaca cgtccacgag cacagtctac 240
atggagctga gcagcctgag atctgaggac acggcggtgt actactgcgc tagaagtatt 300
agtggcaaac gaggtggcga ttactacggc atggatgttt ggggccaggg aacaactgtc 360
accgtctcct cagcctccac caagggccca tcggtcttcc ccctggcgcc ctgctccagg 420
agcacctccg agagcacagc cgccctgggc tgcctggtca aggactactt ccccgaaccg 480
gtgacggtgt cgtggaactc aggcgccctg accagcggcg tgcacacctt cccggctgtc 540
ctacagtcct caggactcta ctccctcagc agcgtggtga ccgtgccctc cagcagcttg 600
ggcacgaaga cctacacctg caacgtagat cacaagccca gcaacaccaa ggtggacaag 660
agagttgagt ccaaatatgg tcccccatgc ccaccctgcc cagcacctga gttcctgggg 720
ggaccatcag tcttcctgtt ccccccaaaa cccaaggaca ctctcatgat ctcccggacc 780
cctgaggtca cgtgcgtggt ggtggacgtg agccaggaag accccgaggt ccagttcaac 840
tggtacgtgg atggcgtgga ggtgcataat gccaagacaa agccgcggga ggagcagttc 900
aacagcacgt accgtgtggt cagcgtcctc accgtcctgc accaggactg gctgaacggc 960
aaggagtaca agtgcaaggt ctccaacaaa ggcctcccgt cctccatcga gaaaaccatc 1020
tccaaagcca aagggcagcc ccgagagcca caggtgtaca ccctgccccc atcccaggag 1080
gagatgacca agaaccaggt cagcctgacc tgcctggtca aaggcttcta ccccagcgac 1140
atcgccgtgg agtgggagag caatgggcag ccggagaaca actacaagac cacgcctccc 1200
gtgctggact ccgacggctc cttcttcctc tacagcaggc tcaccgtgga caagagcagg 1260
tggcaggagg ggaatgtctt ctcatgctcc gtgatgcatg aggctctgca caaccactac 1320
acacagaagt ccctctccct gtctctgggt aaatga 1356
SEQ ID NO: 10
QVQLVQSGAE VKEPGASVKV SCKASGYTFA EYYMHWVRQA PGQGLEWMGA INPSGSHTSY 60
AQKFQGRVTM TRDTSTSTVY MELSSLRSED TAVYYCARSI SGKRGGDYYG MDVWGQGTTV 120
TVSSASTKGP SVFPLAPCSR STSESTAALG CLVKDYFPEP VTVSWNSGAL TSGVHTFPAV 180
LQSSGLYSLS SVVTVPSSSL GTKTYTCNVD HKPSNTKVDK RVESKYGPPC PPCPAPEFLG 240
GPSVFLFPPK PKDTLMISRT PEVTCVVVDV SQEDPEVQFN WYVDGVEVHN AKTKPREEQF 300
NSTYRVVSVL TVLHQDWLNG KEYKCKVSNK GLPSSIEKTI SKAKGQPREP QVYTLPPSQE 360
EMTKNQVSLT CLVKGFYPSD IAVEWESNGQ PENNYKTTPP VLDSDGSFFL YSRLTVDKSR 420
WQEGNVFSCS VMHEALHNHY TQKSLSLSLG K 451
SEQ ID NO: 11
gacatccaga tgacccagtc tccttccacc ctgtctgcat ctgtaggaga cagagtcacc 60
atcacttgcc gggccagtca gagtattagt agctggttgg cctggtatca gcagaaacca 120
gggaaagccc ctaagctcct gatctataaa gcctccagct tggaaagtgg ggtcccatca 180
aggttcagcg gcagtggatc tgggacagaa ttcactctca ccatcagcag cctgcagcct 240
gatgattttg caacttatta ctgccagcag tacagcatct tccctttcac ttttggcgga 300
gggaccaagg ttgagatcaa acgaactgtg gctgcaccat ctgtcttcat cttcccgcca 360
tctgatgagc agttgaaatc tggaactgcc tctgttgtgt gcctgctgaa taacttctat 420
cccagagagg ccaaagtaca gtggaaggtg gataacgccc tccaatcggg taactcccag 480
gagagtgtca cagagcagga cagcaaggac agcacctaca gcctcagcag caccctgacg 540
ctgagcaaag cagactacga gaaacacaaa gtctacgcct gcgaagtcac ccatcagggc 600
ctgagctcgc ccgtcacaaa gagcttcaac aggggagagt gttag 645
SEQ ID NO: 12
DIQMTQSPST LSASVGDRVT ITCRASQSIS SWLAWYQQKP GKAPKLLIYK ASSLESGVPS 60
RFSGSGSGTE FTLTISSLQP DDFATYYCQQ YSIFPFTFGG GTKVEIKRTV AAPSVFIFPP 120
SDEQLKSGTA SVVCLLNNFY PREAKVQWKV DNALQSGNSQ ESVTEQDSKD STYSLSSTLT 180
LSKADYEKHK VYACEVTHQG LSSPVTKSFN RGEC 214
SEQ ID NO: 13
caggtgcagc tggtggagtc tgggggaggc gtagtccagc ctgggaggtc cctgagactc 60
tcctgtgcag cgtctggatt caccttcagt gcgtattgga tgcactgggt ccgccaggct 120
ccaggcaagg ggctggagtg ggtggcagtt atatcgtatg atggaagtaa taaatactat 180
gcagactccg tgaagggccg attcaccatc tccagagacc attccaagaa cacgctgtat 240
ctgcaaatga acagcctgag agccgaggac acggcggtgt actactgcgc aaaaggggac 300
gcctacgact cctggaggat actactcggc ggagattact acggcatgga tgtttggggc 360
cagggaacaa ctgtcaccgt ctcctca 387
SEQ ID NO: 14
QVQLVESGGG VVQPGRSLRL SCAASGFTFS AYWMHWVRQA PGKGLEWVAV ISYDGSNKYY 60
ADSVKGRFTI SRDHSKNTLY LQMNSLRAED TAVYYCAKGD AYDSWRILLG GDYYGMDVWG 120
QGTTVTVSS 129
SEQ ID NO: 15
ggattcacct tcagtgcgta ttgg 24
SEQ ID NO: 16
GFTFSAYW 8
SEQ ID NO: 17
atatcgtatg atggaagtaa taaa 24
SEQ ID NO: 18
ISYDGSNK 8
SEQ ID NO: 19
gcaaaagggg acgcctacga ctcctggagg atactactcg gcggagatta ctacggcatg 60
gatgtt 66
SEQ ID NO: 20
AKGDAYDSWR ILLGGDYYGM DV 22
SEQ ID NO: 21
gaaattgtgt tgacgcagtc tccaggcacc ctgtctttgt ctccagggga aagagccacc 60
ctctcctgca gggccagtca gagtgttagc agcagctact tagcctggta ccagcagaaa 120
cctggccagg ctcccaggcc cctcatctat ggcgcatcca gcagggccac tggcatccca 180
gacagattca gtggcagtgg gtctgggaca gacttcactc tcaccatcag cagactggag 240
cctgaagatt ttgcagtgta ttactgtcag cagggggggg acgcccctcc ttacactttc 300
ggcggaggga ccaaggttga gatcaaa 327
SEQ ID NO: 22
EIVLTQSPGT LSLSPGERAT LSCRASQSVS SSYLAWYQQK PGQAPRPLIY GASSRATGIP 60
DRFSGSGSGT DFTLTISRLE PEDFAVYYCQ QGGDAPPYTF GGGTKVEIK 109
SEQ ID NO: 23
cagagtgtta gcagcagcta c 21
SEQ ID NO: 24
QSVSSSY 7
SEQ ID NO: 25
GGCGCATCC 9
SEQ ID NO: 26
GAS 3
SEQ ID NO: 27
cagcaggggg gggacgcccc tccttacact 30
SEQ ID NO: 28
QQGGDAPPYT 10
SEQ ID NO: 29
cagctgcagc tgcaggagtc gggcccagga ctggtgaagc cttcggagac cctgtccctc 60
acctgcactg tctctggtgg ctccatcacg agtagtagtt actactgggc gtggatccgc 120
cagcccccag ggaaggggct ggagtggatt gggaagatct attatagtgg gagcacccat 180
tacaacccgt ccctcaagag tcgagtcacc atatccgtag acacgtccaa gaaccagttc 240
tccctgaagt tgagttctgt gaccgccgca gacacggcgg tgtactactg cgccagagtt 300
ggaggacacg gctacggaga ctccgcaggg ttagccttcg atatctgggg tcagggtaca 360
atggtcaccg tctcctca 378
SEQ ID NO: 30
QLQLQESGPG LVKPSETLSL TCTVSGGSIT SSSYYWAWIR QPPGKGLEWI GKIYYSGSTH 60
YNPSLKSRVT ISVDTSKNQF SLKLSSVTAA DTAVYYCARV GGHGYGDSAG LAFDIWGQGT 120
MVTVSS 126
SEQ ID NO: 31
ggtggctcca tcacgagtag tagttactac 30
SEQ ID NO: 32
GGSITSSSYY 10
SEQ ID NO: 33
atctattata gtgggagcac c 21
SEQ ID NO: 34
IYYSGST 7
SEQ ID NO: 35
gccagagttg gaggacacgg ctacggagac tccgcagggt tagccttcga tatc 54
SEQ ID NO: 36
ARVGGHGYGD SAGLAFDI 18
SEQ ID NO: 37
gaaattgtgt tgacgcagtc tccaggcacc ctgtctttgt ctccagggga aagagccacc 60
ctctcctgca gggccagtca gagtgttagc agcgccttct tagcctggta ccagcagaaa 120
cctggccagg ctcccaggct cctcatctat ggtgcatcct acaggcacac tggcatccca 180
gacaggttca gtggcagtgg gtctgggaca gacttcactc tcaccatcag cagactggag 240
cctgaagatt ttgcagtgta ttactgtcag cactacggag ttggtcctag gactttcggc 300
ggagggacca aggttgagat caaa 324
SEQ ID NO: 38
EIVLTQSPGT LSLSPGERAT LSCRASQSVS SAFLAWYQQK PGQAPRLLIY GASYRHTGIP 60
DRFSGSGSGT DFTLTISRLE PEDFAVYYCQ HYGVGPRTFG GGTKVEIK 108
SEQ ID NO: 39
cagagtgtta gcagcgcctt c 21
SEQ ID NO: 40
QSVSSAF 7
SEQ ID NO: 41
GGTGCATCC 9
SEQ ID NO: 42
GAS 3
SEQ ID NO: 43
cagcactacg gagttggtcc taggact 27
SEQ ID NO: 44
QHYGVGPRT 9
SEQ ID NO: 45
caggtgcagc tggtgcagtc tggggctgag gtgaaggagc ctggggcctc agtgaaggtt 60
tcctgcaagg catctggata caccttcgcg gagtactata tgcactgggt gcgacaggcc 120
cctggacaag ggcttgagtg gatgggagcg attaacccta gtggtagtca tacaagctac 180
gcacagaagt tccagggcag agtcaccatg accagggaca cgtccacgag cacagtctac 240
atggagctga gcagcctgag atctgaggac acggcggtgt actactgcgc tagaagtatt 300
agtggcaaac gaggtggcga ttactacggc atggatgttt ggggccaggg aacaactgtc 360
accgtctcct ca 372
SEQ ID NO: 46
QVQLVQSGAE VKEPGASVKV SCKASGYTFA EYYMHWVRQA PGQGLEWMGA INPSGSHTSY 60
AQKFQGRVTM TRDTSTSTVY MELSSLRSED TAVYYCARSI SGKRGGDYYG MDVWGQGTTV 120
TVSS 124
SEQ ID NO: 47
ggatacacct tcgcggagta ctat 24
SEQ ID NO: 48
GYTFAEYY 8
SEQ ID NO: 49
attaacccta gtggtagtca taca 24
SEQ ID NO: 50
INPSGSHT 8
SEQ ID NO: 51
gctagaagta ttagtggcaa acgaggtggc gattactacg gcatggatgt t 51
SEQ ID NO: 52
ARSISGKRGG DYYGMDV 17
SEQ ID NO: 53
gacatccaga tgacccagtc tccttccacc ctgtctgcat ctgtaggaga cagagtcacc 60
atcacttgcc gggccagtca gagtattagt agctggttgg cctggtatca gcagaaacca 120
gggaaagccc ctaagctcct gatctataaa gcctccagct tggaaagtgg ggtcccatca 180
aggttcagcg gcagtggatc tgggacagaa ttcactctca ccatcagcag cctgcagcct 240
gatgattttg caacttatta ctgccagcag tacagcatct tccctttcac ttttggcgga 300

gggaccaagg ttgagatcaa a 321
SEQ ID NO: 54
DIQMTQSPST LSASVGDRVT ITCRASQSIS SWLAWYQQKP GKAPKLLIYK ASSLESGVPS 60
RFSGSGSGTE FTLTISSLQP DDFATYYCQQ YSIFPFTFGG GTKVEIK 107
SEQ ID NO: 55
cagagtatta gtagctgg 18
SEQ ID NO: 56
QSISSW 6
SEQ ID NO: 57
AAAGCCTCC 9
SEQ ID NO: 58
KAS 3
SEQ ID NO: 59
cagcagtaca gcatcttccc tttcact 27
SEQ ID NO: 60
QQYSIFPFT 9
SEQ ID NO: 61
MVDGVMILPV LIMIALPSPS MEDEKPKVNP KLYMCVCEGL SCGNEDHCEG QQCFSSLSIN 60
DGFHVYQKGC FQVYEQGKMT CKTPPSPGQA VECCQGDWCN RNITAQLPTK GKSFPGTQNF 120
HLEVGLIILS VVFAVCLLAC LLGVALRKFK RRNQERLNPR DVEYGTIEGL ITTNVGDSTL 180
ADLLDHSCTS GSGSGLPFLV QRTVARQITL LECVGKGRYG EVWRGSWQGE NVAVKIFSSR 240
DEKSWFRETE LYNTVMLRHE NILGFIASDM TSRHSSTQLW LITHYHEMGS LYDYLQLTTL 300
DTVSCLRIVL SIASGLAHLH IEIFGTQGKP AIAHRDLKSK NILVKKNGQC CIADLGLAVM 360
HSQSTNQLDV GNNPRVGTKR YMAPEVLDET IQVDCFDSYK RVDIWAFGLV LWEVARRMVS 420
NGIVEDYKPP FYDVVPNDPS FEDMRKVVCV DQQRPNIPNR WFSDPTLTSL AKLMKECWYQ 480
NPSARLTALR IKKTLTKIDN SLDKLKTDC 509
SEQ ID NO: 62
MVDGVMILPV LMMMAFPSPS VEDEKPKVNQ KLYMCVCEGL SCGNEDHCEG QQCFSSLSIN 60
DGFHVYQKGC FQVYEQGKMT CKTPPSPGQA VECCQGDWCN RNITAQLPTK GKSFPGTQNF 120
HLEVGLIILS VVFAVCLLAC ILGVALRKFK RRNQERLNPR DVEYGTIEGL ITTNVGDSTL 180
AELLDHSCTS GSGSGLPFLV QRTVARQITL LECVGKGRYG EVWRGSWQGE NVAVKIFSSR 240
DEKSWFRETE LYNTVMLRHE NILGFIASDM TSRHSSTQLW LITHYHEMGS LYDYLQLTTL 300
DTVSCLRIVL SIASGLAHLH IEIFGTQGKS AIAHRDLKSK NILVKKNGQC CIADLGLAVM 360
HSQSTNQLDV GNNPRVGTKR YMAPEVLDET IQVDCFDSYK RVDIWAFGLV LWEVARRMVS 420
NGIVEDYKPP FYDVVPNDPS FEDMRKVVCV DQQRPNIPNR WFSDPTLTSL AKLMKECWYQ 480
NPSARLTALR IKKTLTKIDN SLDKLKTCD 509
SEQ ID NO: 63
MEDEKPKVNP KLYMCVCEGL SCGNEDHCEG QQCFSSLSIN DGFHVYQKGC FQVYEQGKMT 60
CKTPPSPGQA VECCQGDWCN RNITAQLPTK GKSFPGTQNF HLEEQKLISE EDLGGEQKLI 120
SEEDLHHHHH H 131
SEQ ID NO: 64
MEDEKPKVNP KLYMCVCEGL SCGNEDHCEG QQCFSSLSIN DGFHVYQKGC FQVYEQGKMT 60
CKTPPSPGQA VECCQGDWCN RNITAQLPTK GKSFPGTQNF HLEEPRGPTI KPCPPCKCPA 120
PNLLGGPSVF IFPPKIKDVL MISLSPIVTC VVVDVSEDDP DVQISWFVNN VEVHTAQTQT 180
HREDYNSTLR VVSALPIQHQ DWMSGKEFKC KVNNKDLPAP IERTISKPKG SVRAPQVYVL 240
PPPEEEMTKK QVTLTCMVTD FMPEDIYVEW TNNGKTELNY KNTEPVLDSD GSYFMYSKLR 300
VEKKNWVERN SYSCSVVHEG LHNHHTTKSF SRTPGK 336
SEQ ID NO: 65
VEDEKPKVNQ KLYMCVCEGL SCGNEDHCEG QQCFSSLSIN DGFHVYQKGC FQVYEQGKMT 60
CKTPPSPGQA VECCQGDWCN RNITAQLPTK GKSFPGTQNF HLEEQKLISE EDLGGEQKLI 120
SEEDLHHHHH H 131

1. An isolated antibody or antigen-binding fragment thereof that binds specifically to activin A receptor type 1 (ACVR1) protein and/or a mutant thereof, wherein the antibody or antigen-binding fragment thereof interacts with one or more amino acids contained within the extracellular domain of ACVR1 (amino acids 21-123 of SEQ ID NO: 61), and wherein the antibody or antigen-binding fragment thereof binds to cells expressing full length ACVR1 protein and/or a mutant thereof.

2. The isolated antibody or antigen-binding fragment of claim 1, wherein the full-length ACVR1 protein or mutant thereof is a full length human ACVR protein or mutant thereof.

3. The isolated antibody or antigen-binding fragment thereof of claim 2, wherein the full-length human ACVR1 protein comprises amino acids 21-509 of SEQ ID NO: 61.

4. The isolated antibody or antigen-binding fragment thereof of claim 1, wherein the mutant ACVR1 protein comprises a mutation selected from the group consisting of ACVR1 L196P, delP197_F198insL, R202I, R206H, Q207E, R258S, R258G, G325A, G328E, G328R, G328W, G356D, and R375P of SEQ ID NO: 61.

5. The isolated antibody or antigen-binding fragment of claim 4, wherein the isolated antibody or antigen-binding fragment thereof binds to ACVR1(R206H) protein and inhibits ACVR1(R206H)-mediated bone morphogenetic protein (BMP) signal transduction.

6. An isolated antibody or antigen-binding fragment thereof that binds specifically to an ACVR1 protein, wherein the antibody or antigen-binding fragment thereof: (i) binds to cells expressing human ACVR1; and/or (ii) binds to ACVR1 and inhibits ACVR1-mediated bone morphogenetic protein (B1VIP) signal transduction.

7. The antibody or antigen-binding fragment thereof of claim 1, wherein the antibody is a fully human monoclonal antibody.

8. The antibody or antigen-binding fragment thereof of claim 1, wherein the antibody has one or more properties selected from the group consisting of: (a) is a fully human monoclonal antibody; (b) binds to human ACVR1 extracellular domain fused to mFc (SEQ ID NO: 64) at 37° C. with a dissociation constant (K_{D}) of less than 15 nM, less than 10 nM, less than less than 5 nM, less than 3 nM, less than 2 nM, less than 1 nM, less than 0.5 nM, less than 0.3, less than 0.2 nM, or less than 0.1 nM as measured in a surface plasmon resonance assay; (c) binds to human ACVR1 extracellular domain fused to myc-myc-hexahistag (e.g., SEQ ID NO: 63) at 37° C. with a K_{D} of less than 50 nM, less than 10 nM, less than 5 nM, less than 3 nM, less than 2 nM, less than 1 nM less than 0.5 nM as measured in a surface plasmon resonance assay; (d) binds to mouse ACVR1 extracellular domain fused to myc-myc-hexahistag (e.g., SEQ ID NO: 65) at 37° C. with a K_{D} of less than 50 nM, less than 10 nM, less than 5 nM, less than 3 nM, less than 2 nM, less than 1 nM less than 0.5 nM as measured in a surface plasmon resonance assay; (e) binds to mouse ACVR1 extracellular domain fused to mFc at 37° C. with a K_{D} of less than 10 nM, less than 5 nM, less than 3 nM, less than 2 nM, less than 1 nM less than 0.5 nM, less than 0.2 nM, or less than 0.1 nM; (f) binds to cells expressing human ACVR1 protein or human ACVR (R206H) protein; (g) inhibits activation of cells expressing human ACVR1(R206H) by human Activin A with a IC₅₀ of with a IC₅₀ of less than 10 nM, less than 5 nM, less than 3 nM, less than 2 nM, or less than 1 nM, or less as measured in a cell-based bioassay; (h) inhibits activation of cells expressing human ACVR1(R206H) by human BMP7 with a IC₅₀ of with a IC₅₀ of less than 10 nM, less than 5 nM, less than 3 nM, less than 2 nM, or less than 1 nM, or less as measured in a cell-based bioassay; and (i) comprises a HCVR comprising an amino acid sequence selected from the group consisting of HCVR sequence listed in Table 1 and a LCVR comprising an amino acid sequence selected from the group consisting of LCVR sequences listed in Table 1.

9. The antibody or antigen-binding fragment of claim 1, wherein the antibody or antigen-binding fragment comprises three heavy chain complementarity determining regions (CDRs) (HCDR1, HCDR2 and HCDR3) contained within a heavy chain variable region (HCVR); and three light chain CDRs (LCDR1, LCDR2 and LCDR3) contained within a light chain variable region (LCVR), wherein the HCVR has an amino acid sequence selected from the group consisting of HCVR sequences listed in Table 1.

10. The antibody or antigen-binding fragment thereof of claim 9 comprising a LCVR having an amino acid sequence selected from the group consisting of LCVR sequences listed in Table 1.

11. The antibody or antigen-binding fragment thereof of claim 9 comprising one or more of the group consisting of:
(a) a HCDR1 domain having an amino acid sequence selected from the group consisting of SEQ ID NOs: 16, 32, and 48;
(b) a HCDR2 domain having an amino acid sequence selected from the group consisting of SEQ ID NOs: 18, 34, and 50;
(c) a HCDR3 domain having an amino acid sequence selected from the group consisting of SEQ ID NOs: 20, 36, and 52;
(d) a LCDR1 domain having an amino acid sequence selected from the group consisting of SEQ ID NOs: 24, 40, and 56;
(e) a LCDR2 domain having an amino acid sequence selected from the group consisting of SEQ ID NOs: 26, 42, and 58;
and
(f) a LCDR3 domain having an amino acid sequence selected from the group consisting of SEQ ID NOs: 28, 44, and 60.

12. The antibody or antigen-binding fragment thereof of claim 11, comprising a HCVR/LCVR amino acid sequence pair selected from the group consisting of SEQ ID NOs:
14/22, 30/38, and 46/54.

13. The antibody or antigen-binding fragment thereof of claim 12, comprising CDRs selected from the group consisting of: (a) SEQ ID NOs: 16, 18, 20, 24, 26, and 28; (b) SEQ ID NOs: 32, 34, 36, 40, 42, and 44; and (c) SEQ ID NOs:48, 50, 52, 56, 58, and 60.

14. The antibody or antigen-binding fragment thereof of claim 13, comprising a HCVR/LCVR amino acid sequence pair selected from the group consisting of SEQ ID NOs:
30/38, and 46/54.

15. An antibody or antigen-binding fragment thereof that binds to ACVR1, wherein the antibody or antigen-binding fragment comprises three heavy chain CDRs (HCDR1, HCDR2 and HCDR3) contained within a HCVR and three light chain CDRs (LCDR1, LCDR2 and LCDR3) contained within a LCVR; wherein the HCVR comprises:
(i) an amino acid sequence selected from the group consisting of SEQ ID NOs: 14, 30, and 46;
(ii) an amino acid sequence having at least 90% identity to the amino acid sequence selected from the group consisting of SEQ ID NOs: 14, 30, and 46;
(iii) an amino acid sequence having at least 95% identity to the amino acid sequence selected from the group consisting of SEQ ID NOs: 14, 30, and 46; or (iv) an amino acid sequence selected from the group consisting of SEQ ID NOs: 14, 30, and 46, said amino acid sequence having no more than 12 amino acid substitutions, and the LCVR comprises:
(a) an amino acid sequence selected from the group consisting of SEQ ID NOs: 22, 38, and 54;
(b) an amino acid sequence having at least 90% identity to the amino acid sequence selected from the group consisting of SEQ ID NOs: 22, 38, and 54;
(c) an amino acid sequence having at least 95% identity to the amino acid sequence selected from the group consisting of SEQ ID NOs: 22, 38, and 54; or
(d) an amino acid sequence selected from the group consisting of SEQ ID NOs: 22, 38, and 54, said amino acid sequence having no more than 10 amino acid substitutions.

16. The antibody or antigen-binding fragment thereof of claim 15 comprising a HCVR having an amino acid sequence selected from the group consisting of SEQ ID NOs: 14, 30, and 46.

17. The antibody or antigen-binding fragment thereof of claim 15 comprising a LCVR having an amino acid sequence selected from the group consisting of SEQ ID NOs: 22, 38, and 54.

18. The antibody or antigen-binding fragment thereof of claim 15 comprising three CDRs contained within a HCVR selected from the group consisting of SEQ ID NOs: 14, 30, and 46; and three CDRs contained within a LCVR selected from the group consisting of SEQ ID NOs: 22, 38, and 54.

19. The antibody or antigen-binding fragment of claim 15 comprising a HCVR/LCVR amino acid sequence pair selected from the group consisting of SEQ ID NOs:14/22, 30/38, and 46/54.

20. The antibody or antigen-binding fragment thereof of claim 15 comprising:
(a) a HCDR1 domain having an amino acid sequence selected from the group consisting of SEQ ID NOs: 16, 32, and 48;
(b) a HCDR2 domain having an amino acid sequence selected from the group consisting of SEQ ID NOs: 18, 34, and 50;
(c) a HCDR3 domain having an amino acid sequence selected from the group consisting of SEQ ID NOs: 20, 36, and 52;
(d) a LCDR1 domain having an amino acid sequence selected from the group consisting of SEQ ID NOs: 24, 40, and 56;
(e) a LCDR2 domain having an amino acid sequence selected from the group consisting of SEQ ID NOs: 26, 42, and 58; and
(f) a LCDR3 domain having an amino acid sequence selected from the group consisting of SEQ ID NOs: 28, 44, and 60.

21. The antibody or antigen-binding fragment thereof of claim 20 comprising CDRs selected from the group consisting of: (a) SEQ ID NOs: 16, 18, 20, 24, 26, and 28; (b) SEQ ID NOs: 32, 34, 36, 40, 42, and 44; and (c) SEQ ID NOs: 48, 50, 52, 56, 58, and 60.

22. The antibody or antigen-binding fragment thereof of claim 21 comprising a HCVR/LCVR amino acid sequence pair selected from the group consisting of SEQ ID NOs:
14/22, 30/38, and 46/54.

23. An isolated monoclonal antibody or antigen-binding fragment thereof that inhibits ACVR-mediated and/or ACVR1(R206H)-mediated bone morphogenetic protein (BMP) signal transduction comprising three CDRs of a HCVR, wherein the HCVR has an amino acid sequence selected from the group consisting of SEQ ID NOs: 14, 30, and 46; and three CDRs of a LCVR, wherein the LCVR has an amino acid sequence selected from the group consisting of SEQ ID NOs: 22, 38, and 54.

24. An antibody or antigen-binding fragment thereof that competes for binding to ACVR1 with an antibody or antigen-binding fragment thereof of claim 1.

25. An antibody or antigen-binding fragment thereof that binds to the same epitope as an antibody or antigen-binding fragment thereof of claim 1.

26. A pharmaceutical composition comprising an isolated antibody or antigen-binding fragment thereof that binds to ACVR1 according to claim 1 and a pharmaceutically acceptable carrier or diluent.

27. An isolated polynucleotide molecule comprising a polynucleotide sequence that encodes a HCVR of an antibody as set forth in claim 1.

28. An isolated polynucleotide molecule comprising a polynucleotide sequence that encodes a LCVR of an antibody as set forth in claim 1.

29. A vector comprising the polynucleotide sequence of claim 27 and/or the polynucleotide sequence of claim 27.

30. A host cell expressing the vector of claim 29.

31. A method of producing an anti-ACVR1 antibody or antigen-binding fragment thereof, comprising growing the host cell of claim 30 under conditions permitting production of the antibody or fragment, and recovering the antibody or fragment so produced.

32. The method of claim 31, further comprising formulating the antibody or antigen-binding fragment thereof as a pharmaceutical composition comprising an acceptable carrier.

33. A method of treating, preventing, ameliorating, or reducing recurrence of at least one symptom or indication of a ACVR1-associated disease or disorder, the method comprising administering a pharmaceutical composition comprising a therapeutically effective amount of an antibody or antigen-binding fragment thereof of claim 1 to a subject in need thereof.

34. The method of claim 33, wherein the ACVR1-associated disease or disorder is selected from the group consisting of heterotopic ossification, trauma-induced heterotopic ossification, ectopic ossification, bone dysplasia, anemia, and diffuse intrinsic pontine glioma.

35. The method of claim 33, wherein the pharmaceutical composition is administered prophylactically or therapeutically to the subject in need thereof.

36. The method of claim 33, wherein the pharmaceutical composition is administered in combination with a second therapeutic agent.

37. The method of claim 36, wherein the second therapeutic agent is selected from the group consisting of an anti-Activin A inhibitor, anti-BMP7 antibody or antigen binding fragment thereof, anti-ACVR2 antibody or antigen-binding fragment thereof, anti-inflammatory drugs, steroids, bisphosphonates, muscle relaxants, and retinoic acid receptor (RAR) gamma agonists, a lifestyle modification, and a dietary supplement.

38. The method of claim 33, wherein the pharmaceutical composition is administered subcutaneously, intravenously, intradermally, intraperitoneally, intramuscularly, or intracerebroventricularly.
